(19) United States
(12) Patent Application Publication
(10) Pub. No.: US 2024/0240257 A1
(43) Pub. Date: Jul. 18, 2024

(54) COMPOSITIONS AND METHODS FOR IMPROVED 5-HYDROXYMETHYLATED CYTOSINE RESOLUTION IN NUCLEIC ACID SEQUENCING

(71) Applicant: Freenome Holdings, Inc., South San Francisco, CA (US)

(72) Inventors: Eric ARIAZI, Redwood City, CA (US); Paula ESQUETINI, Foster City, CA (US); Aneesha TEWARI, San Francisco, CA (US); David WEINBERG, Mill Valley, CA (US)

(21) Appl. No.: 18/417,054

(22) Filed: Jan. 19, 2024

Related U.S. Application Data
(63) Continuation of application No. PCT/US2022/037557, filed on Jul. 19, 2022.
(60) Provisional application No. 63/223,661, filed on Jul. 20, 2021.

(51) Int. Cl. C12Q 1/6886; C12Q 1/6806; C12Q 1/6811; C12Q 1/6874

(52) U.S. Cl. CPC C12Q 1/6886; C12Q 1/6811; C12Q 1/6874; C12Q 1/6806; C12Q 2600/154

(57) ABSTRACT

The present disclosure provides oligonucleotide adapter compositions, methods, and systems for improved resolution of 5hmC sequencing useful for improving nucleic acid sequencing library quality and nucleic acid methylation profiling. Also provided are methods of applying the improved oligonucleotide adapters and sequencing methods for machine learning classifier generation, and detecting cell proliferative disorders such as cancer. Methods of applying targeted nucleic acid enrichment with methods of applying the improved oligonucleotide adapters and sequencing methods for improving nucleic acid sequencing library quality and nucleic acid methylation profiling are also provided.

### CROSS-REFERENCE TO RELATED APPLICATIONS

This application is a continuation of International Patent Application No. PCT/US2022/037557, filed Jul. 19, 2022, which claims the benefit of U.S. Provisional Patent Application No. 63/223,661, filed Jul. 20, 2021, each of which is incorporated by reference herein in its entirety.

### FIELD

The present disclosure relates generally to improved adapters and methods for performing methylation analysis of nucleic acid sequences. The present disclosure relates to sequencing adapters and methods of use to improve the sequencing resolution for 5-hydroxymethylated cytosine that may be useful for nucleic acid methylation pattern analysis.

### BACKGROUND

DNA methylation occurs predominantly at cytosines in CpG dinucleotides and acts as an epigenetic mark with functional roles in gene regulation. Methylation marks are heritable, and their genome-wide profiles differ from tissue to tissue. In cancer, gene-specific methylation profiles may become aberrant, but retain similarity to the tissue of origin which make methylation marks useful biomarkers for cancer diagnosis and prognosis.

5-methylcytosine (5mC) and 5-hydroxymethylcytosine (5hmC) are two forms of epigenetic modification at the 5-carbon position of cytosine and associated with gene silencing and activation, respectively. These methylation marks provide various types of information that may be used to build classification models to infer the presence of cancer. High quality sequence information is desirable to produce classification models to infer disease with high sensitivity and specificity, and such information may be lost during sample processing and sequencing thereby impacting accuracy of such models.

Several sequencing methods may be used to identify 5hmC; however, these methods have advantages and disadvantages that impact adoption for commercial screening and diagnostic use, e.g., lack of nucleotide-resolution, false positive 5hmC calls, high sample input requirement, inferring by subtraction rather than direct readout, and the quality of sequencing libraries produced for sequencing from a nucleic acid sample. Therefore, tools and methods may be needed to improve the quality of hydroxymethylation status information provided from nucleic acid sequencing that may be useful in classification models of disease diagnosis, prognosis, and progression.

### SUMMARY

The present disclosure provides compositions, methods, and systems directed to improved detection of hydroxymethylated cytosine during nucleic acid sequencing. Methods and compositions used in such methods described herein may be used to overcome the limitations of unmethylated and methylated cytosine conversion methods such as TAB-seq and ACE-seq used prior to nucleic acid sequencing. In various aspects, using modified adapters containing 5hmC, or a combination of 5-(β-glucosyloxymethyl)cytosine (5gmC) and 5-carboxycytosine (5caC) or 5-carboxymethylcytosine (5cxmC), and ligation of such adapters to nucleic acid fragments in a biological sample, may improve the resolution of hydroxymethylation sequence information in the sample.

In an aspect, the present disclosure provides oligonucleotide adapters that comprise one or more 5hmC, 5gmC, 5caC, 5cxmC nucleotides, or a combination thereof, and no cytosine nucleotides, which may be used in ligation to a nucleic acid molecule in a biological sample for nucleic acid sequencing. In some embodiments, there are no cytosine nucleotides in a flow cell binding region or primer binding site of the adapters. In some embodiments, cytosine nucleotides exist in a UMI portion of the adapter, but not in the non-UMI portion of the adapter. In some embodiments, cytosine nucleotides exist in a primer binding site portion of the adapter, but not in the non-primer binding site portion of the adapter. The oligonucleotides are capable of ligating to a nucleic acid sequence before treatment with conditions necessary to convert unmethylated and methylated cytosines in the nucleic acid sequence to uracil and are capable of hybridizing to primers for downstream amplification and sequencing methods.

In another aspect, the present disclosure provides a method for providing hydroxymethylation state data of nucleic acids in a biological sample, the method comprising:
a) obtaining the biological sample containing the nucleic acids;
b) ligating oligonucleotide adapters to at least a portion of the nucleic acids in the biological sample, wherein the oligonucleotide adapters comprise 5hmC nucleotides, 5gmC nucleotides, 5caC nucleotides, 5cxmC nucleotides, or a combination thereof, thereby generating ligated nucleic acids;
c) subjecting at least a portion of the ligated nucleic acids or a derivative thereof to a conversion condition that converts unmethylated and methylated cytosine nucleotides but not hydroxymethylated cytosine nucleotides in of the ligated nucleic acids into uracil nucleotides, thereby generating converted nucleic acids; and
d) sequencing at least a portion of the converted nucleic acids to obtain a nucleic acid sequence of the converted nucleic acids to provide the hydroxymethylation state data of the nucleic acids.

In some embodiments, the oligonucleotide adapters do not comprise cytosine nucleotides in flow cell binding regions or primer binding sites of the oligonucleotide adapters.

In some embodiments, the method further comprises subjecting at least a portion of the ligated nucleic acids to glucosylation by β-glucosyltransferase (β-GT)/UDP-glucose to convert 5hmC nucleotides into 5gmC nucleotides after b) or prior to c).

In some embodiments, the conversion condition comprises bisulfite treatment, enzymatic treatment, or a combination thereof.

In some embodiments, the oligonucleotide adapters comprise 5hmC nucleotides.

In some embodiments, the oligonucleotide adapters comprise 5gmC and 5caC nucleotides.

In some embodiments, the oligonucleotide adapters comprise 5gmC nucleotides, 5caC nucleotides, 5cxmC nucleotides, or a combination thereof.

In some embodiments, the conversion condition comprises treatment with β-GT, a cytosine dioxygenase enzyme, carboxymethyltransferase, apolipoprotein B mRNA editing catalytic polypeptide-like protein (AID/APOBEC), or a combination thereof.

In some embodiments, the cytosine dioxygenase enzyme comprises ten eleven translocation protein 1 (TET1), ten eleven translocation protein 2 (TET2), ten eleven translocation protein 3 (TET3), or a functional variant thereof.

In some embodiments, the method further comprises treating the oligonucleotide adapters with a TET enzyme after a) or prior to b).

In some embodiments, the method further comprises performing a sequence enrichment after b) or prior to c).

In some embodiments, the sequence enrichment comprises a target capture hybridization.

In some embodiments, at least a portion of the ligated nucleic acids are amplified prior to the sequencing.

In some embodiments, the method further comprises amplifying at least a portion of the ligated nucleic acids prior to the sequencing.

In some embodiments, the method further comprises preparing a nucleic acid sequencing library prior to the amplifying.

In some embodiments, the method further comprises aligning the nucleic acid sequence to a reference genome.

In some embodiments, the oligonucleotide adapters are chemically synthesized using 5hmC phosphoramidites.

In some embodiments, the oligonucleotide adapters comprise 5gmC and 5caC nucleotides, wherein the oligonucleotide adapters are produced at least in part by synthesizing 5mC-containing oligonucleotides using phosphoramidite chemistry and enzymatically treating the 5mC-containing oligonucleotides with a TET enzyme and β-GT/UDP-glucose.

In some embodiments, the oligonucleotide adapters are synthesized using terminal deoxynucleotidyl transferase (TdT)-mediated enzymatic oligonucleotide synthesis.

In some embodiments, the method further comprises methylating unmethylated cytosine nucleotides in the 5mC-containing oligonucleotides using SAM-dependent C5-methyltransferase (C5-MT) or another DNA cytosine-5 methyltransferase.

In some embodiments, the method further comprises ligating the oligonucleotide adapters to at least a portion of nucleic acids isolated from a biological sample.

In some embodiments, the oligonucleotide adapters are synthesized using an enzymatic oligonucleotide synthesis technique.

In some embodiments, the biological sample comprises cell-free DNA (cfDNA).

In some embodiments, the nucleic acids are cfDNA.

In some embodiments, the biological sample is obtained or derived from an individual, the hydroxymethylation state data are associated with an abnormal cell state or disease and provide classification of the individual as having the abnormal cell state or disease.

In some embodiments, the abnormal cell state or disease is stage 1 cancer, stage 2 cancer, stage 3 cancer, or stage 4 cancer.

In some embodiments, the oligonucleotide adapters comprise a unique molecular identifier.

In some embodiments, the biological sample is selected from the group consisting of a bodily fluid, stool, colonic effluent, urine, cerebrospinal fluid, blood plasma, blood serum, whole blood, isolated blood cells, cells isolated from the blood, and a combination thereof.

In some embodiments, the method further comprises optionally featurizing the hydroxymethylation state data, and processing the featurized hydroxymethylation state data using a machine learning model that is trained to classify the biological sample into groups according to predesignated or preselected biological properties.

In some embodiments, the featurized hydroxymethylation state data correspond to properties of the nucleic acid sequence in the biological sample.

In some embodiments, the properties of the nucleic acid sequence are selected from presence or absence of pre-cancer, cancer or a stage of cancer, or a prognosis of cancer in the subject.

In another aspect, the present disclosure provides a method for generating oligonucleotide adapters, the method comprising:
a) synthesizing 5mC-containing oligonucleotides at least in part by phosphoramidite chemistry; and
b) contacting the 5mC-containing oligonucleotides with a TET enzyme and β-GT/UDP-glucose to convert 5mC nucleotides into 5gmC or 5caC nucleotides, thereby generating the oligonucleotide adapters.

In some embodiments, the oligonucleotide adapters are synthesized using terminal deoxynucleotidyl transferase (TdT)-mediated enzymatic oligonucleotide synthesis.

In some embodiments, the oligonucleotide adapters comprise 5gmC and 5caC nucleotides.

In some embodiments, the method further comprises methylating unmethylated cytosine nucleotides in the 5mC-containing oligonucleotides using SAM-dependent C5-methyltransferase (C5-MT) or another DNA cytosine-5 methyltransferase.

In some embodiments, the method further comprises ligating the oligonucleotide adapters to at least a portion of nucleic acids isolated from a biological sample.

In another aspect, the present disclosure provides a method for generating oligonucleotide adapters, the method comprising:
synthesizing oligonucleotides containing 5gmC nucleotides, 5caC nucleotides, 5cxmC nucleotides, or a combination thereof, at least in part by phosphoramidite chemistry, thereby generating the oligonucleotide adapters.

In some embodiments, the oligonucleotide adapters are synthesized using an enzymatic oligonucleotide synthesis technique.

In some embodiments, the method further comprises ligating the oligonucleotide adapters to at least a portion of nucleic acids isolated from a biological sample.

In another aspect, the present disclosure provides a method for training a machine learning model to generate a hydroxymethylation profile for nucleic acids in a biological sample, the method comprising:
a) obtaining the biological sample containing the nucleic acids;
b) ligating oligonucleotide adapters to at least a portion of the nucleic acids in the biological sample, wherein the oligonucleotide adapters comprise 5hmC nucleotides, 5gmC nucleotides, 5caC nucleotides, 5cxmC nucleotides, or a combination thereof, thereby generating ligated nucleic acids;
c) subjecting at least a portion of the ligated nucleic acids to a conversion condition that converts unmethylated and methylated cytosine nucleotides in the ligated nucleic acids into uracil nucleotides, thereby generating converted nucleic acids;
d) sequencing at least a portion of the converted nucleic acids to obtain a nucleic acid sequence of the converted nucleic acids to provide hydroxymethylation state data of the nucleic acids; and
e) training the machine learning model to generate the hydroxymethylation profile using the hydroxymethylation state data.

In some embodiments, e) further comprises featurizing the hydroxymethylation state data. In some embodiments, the oligonucleotide adapters do not comprise cytosine nucleotides in flow cell binding regions or primer binding sites in the oligonucleotide adapters.

In some embodiments, the method further comprises subjecting at least a portion of the ligated nucleic acids to glucosylation at least in part by β-GT/UDP-glucose to convert 5hmC nucleotides into 5gmC nucleotides after b) or prior to c).

In some embodiments, the biological sample comprises cell-free DNA (cfDNA).

In another aspect, the present disclosure provides a method for determining a hydroxymethylation profile of cfDNA in a biological sample obtained or derived from an individual, the method comprising:
a) obtaining the biological sample containing the cfDNA;
b) ligating oligonucleotide adapters to at least a portion of the cfDNA in the biological sample, wherein the oligonucleotide adapters comprise 5hmC nucleotides, 5gmC nucleotides, 5caC nucleotides, 5cxmC nucleotides, or a combination thereof, thereby generating ligated cfDNA;
c) subjecting at least a portion of the ligated cfDNA or a derivative thereof to a conversion condition that converts unmethylated and methylated cytosine nucleotides in the ligated cfDNA into uracil nucleotides, thereby generating converted cfDNA;
d) sequencing at least a portion of the converted cfDNA to obtain a nucleic acid sequence of the converted cfDNA to provide the hydroxymethylation state data of the cfDNA; and
e) aligning the nucleic acid sequence of the converted cfDNA to a reference nucleic acid sequence to determine the hydroxymethylation profile of the biological sample.

In some embodiments, the method further comprises amplifying the ligated cfDNA prior to the sequencing.

In some embodiments, the method further comprises preparing a nucleic acid sequencing library prior to the amplifying.

In some embodiments, the oligonucleotide adapters do not comprise cytosine nucleotides in flow cell binding regions or primer binding sites in the oligonucleotide adapters.

In some embodiments, the method further comprises subjecting at least a portion of the ligated cfDNA to glucosylation at least in part by β-GT/UDP-glucose to convert hydroxymethylated cytosine nucleotides into 5gmC nucleotide after b) or prior to c).

In some embodiments, the hydroxymethylation profile is associated with an abnormal cell state or disease and provides classification of the individual as having the abnormal cell state or disease.

In some embodiments, the abnormal cell state or disease is stage 1 cancer, stage 2 cancer, stage 3 cancer, or stage 4 cancer.

In some embodiments, the oligonucleotide adapters comprise a unique molecular identifier.

In some embodiments, the conversion condition comprises using a chemical method, an enzymatic method, or a combination thereof.

In some embodiments, the conversion condition comprises treating with bisulfite, hydrogen sulfite, disulfite, or a combination thereof.

In some embodiments, the biological sample is selected from the group consisting of a bodily fluid, stool, colonic effluent, urine, cerebrospinal fluid, blood plasma, blood serum, whole blood, isolated blood cells, cells isolated from the blood, and a combination thereof.

In another aspect, the present disclosure provides a method for generating a classifier for a biological sample, the method comprising:
a) obtaining the biological sample containing nucleic acids;
b) ligating oligonucleotide adapters to at least a portion of the nucleic acids in the biological sample, wherein the oligonucleotide adapters comprise 5hmC nucleotides, 5gmC nucleotides, 5caC nucleotides, 5cxmC nucleotides, or a combination thereof, thereby generating ligated nucleic acids;
c) subjecting at least a portion of the ligated nucleic acids to a conversion condition that converts unmethylated and methylated cytosine nucleotides in the ligated nucleic acids into uracil nucleotides, thereby generating converted nucleic acids;
d) sequencing at least a portion of the converted nucleic acids to obtain a nucleic acid sequence of the converted nucleic acids to provide hydroxymethylation state data of the nucleic acids; and
e) training a machine learning model to generate the classifier using the hydroxymethylation state data.

In some embodiments, the oligonucleotide adapters do not comprise cytosine nucleotides in flow cell binding regions or primer binding sites in the oligonucleotide adapters.

In some embodiments, the method comprises subjecting at least a portion of the ligated nucleic acids to glucosylation at least in part by β-GT/UDP-glucose to convert hydroxymethylated cytosine nucleotides into 5gmC nucleotides after b) or prior to c).

In another aspect, the present disclosure provides a method for generating a classifier for a biological sample obtained or derived from an individual, the method comprising:
a) obtaining the biological sample containing nucleic acids;
b) ligating oligonucleotide adapters to at least a portion of the nucleic acids in the biological sample, wherein the oligonucleotides adapters comprise 5hmC nucleotides, 5gmC nucleotides, 5caC nucleotides, 5cxmC nucleotides, or a combination thereof and do not comprise cytosine nucleotides, thereby generating ligated nucleic acids;
c) subjecting at least a portion of the ligated nucleic acids to a conversion condition that converts unmethylated and methylated cytosine nucleotides in the ligated nucleic acids into uracil nucleotides, thereby generating converted nucleic acids;
d) sequencing at least a portion of the converted nucleic acids to obtain a nucleic acid sequence of the converted nucleic acids to provide hydroxymethylation state data of the nucleic acids; and
e) training a machine learning model to generate a classifier using the hydroxymethylation state data.

In another aspect, the present disclosure provides a method for detecting a cell proliferative disorder in a subject, the method comprising:
a) obtaining a biological sample containing nucleic acids from the subject;
b) ligating oligonucleotide adapters to at least a portion of the nucleic acids in the biological sample wherein the oligonucleotide adapters comprise 5hmC nucleotides, 5gmC nucleotides, 5caC nucleotides, 5cxmC nucleotides, or a combination thereof, thereby generating ligated nucleic acids;
c) subjecting at least a portion of the ligated nucleic acids to a conversion condition that converts unmethylated and methylated cytosine nucleotides in the ligated nucleic acids into uracil nucleotides, thereby generating converted nucleic acids;
d) sequencing at least a portion of the converted nucleic acids to obtain a nucleic acid sequence of the converted nucleic acids to provide hydroxymethylation state data of the nucleic acids; and
e) processing the hydroxymethylation state data using a machine learning model trained to be capable of distinguishing between healthy subjects and subjects with the cell proliferative disorder to provide an output value associated with a presence or a susceptibility of the cell proliferative disorder, thereby indicating the presence or the susceptibility of the cell proliferative disorder in the subject.

In some embodiments, the adapters do not comprise cytosine nucleotides in flow cell binding regions or primer binding sites in the oligonucleotide adapters.

In some embodiments, the method further comprises subjecting at least a portion of the ligated nucleic acids to glucosylation at least in part by β-GT/UDP-glucose to convert hydroxymethylated cytosine nucleotides into 5gmC nucleotides, after b) or prior to c).

In some embodiments, the cell proliferative disorder comprises colorectal cancer, breast cancer, ovarian cancer, prostate cancer, lung cancer, pancreatic cancer, uterine cancer, liver cancer, esophagus cancer, stomach cancer, thyroid cancer, or bladder cancer.

In some embodiments, the machine learning model is tailored to detect the cell proliferative disorder at a pre-selected sensitivity and specificity.

In some embodiments, the machine learning model classifies the presence or the susceptibility of the cell proliferative disorder at a sensitivity of at least about 80%.

In some embodiments, the conversion condition comprises bisulfite treatment, enzymatic treatment, or a combination thereof.

In some embodiments, the oligonucleotide adapters contain 5hmC nucleotides in place of cytosine nucleotides in flow cell binding regions or primer binding sites in the oligonucleotide adapters.

In some embodiments, the oligonucleotide adapters comprise a mixture of 5gmC nucleotides, 5caC nucleotides, 5cxmC nucleotides, or a combination thereof.

In some embodiments, the conversion condition comprises treatment with β-GT, a cytosine dioxygenase enzyme, carboxymethyltransferase, AID/APOBEC, or a combination thereof.

In some embodiments, the cytosine dioxygenase enzyme comprises TET1, TET2, TET3, or a functional variant thereof.

In some embodiments, the method further comprises treating the oligonucleotide adapters with a TET enzyme after a) or prior to b).

In some embodiments, the method further comprises performing a sequence enrichment after b) or prior to c).

In some embodiments, the sequence enrichment comprises a target capture hybridization.

In some embodiments, the method further comprises amplifying at least a portion of the ligated nucleic acids prior to the sequencing.

In some embodiments, the method further comprises aligning the nucleic acid sequence to a reference genome.

In some embodiments, the method further comprises featurizing the hydroxymethylation state data and processing the featurized hydroxymethylation state data using a machine learning model that is trained to classify the biological sample into groups according to predesignated or preselected biological properties.

In some embodiments, the featurized hydroxymethylation state data correspond to properties of the nucleic acid sequence in the biological sample.

In some embodiments, the properties of the nucleic acid sequence are selected from presence or absence of pre-cancer, cancer or a stage of cancer, or a prognosis of cancer in the subject.

In another aspect, the present disclosure provides a method for monitoring minimal residual disease in a subject previously treated for disease, the method comprising: determining a hydroxymethylation profile as a baseline hydroxymethylation state, and further determining a hydroxymethylation profile at each of one or more predetermined time points, wherein a change in hydroxymethylation profile from the baseline hydroxymethylation state indicates a change in the minimal residual disease status at the baseline hydroxymethylation state in the subject.

In some embodiments, the minimal residual disease is indicated by response to treatment, tumor load, residual tumor post-surgery, relapse, secondary screen, primary screen, or cancer progression.

In some embodiments, the method further comprises determining a response of the subject to treatment.

In some embodiments, the method further comprises monitoring a tumor load in the subject.

In some embodiments, the method further comprises detecting a residual tumor in the subject post-surgery.

In some embodiments, the method further comprises detecting a relapse of the subject.

In some embodiments, the method is performed as a secondary screen for the subject.

In some embodiments, the method is performed as a primary screen for the subject.

In some embodiments, the method further comprises monitoring a cancer progression in the subject.

In another aspect, the present disclosure provides a non-transitory computer-readable medium comprising instructions stored thereon which, when executed by one or more processors, are operable to implement a classifier for classifying subjects as having the cell proliferative disorder or not having the cell proliferative disorder based on hydroxymethylation state data obtained from a nucleic acid library generated using oligonucleotide adapters ligated to nucleic acids in the biological sample, wherein the oligonucleotide adapters comprise 5hmC nucleotides, 5gmC nucleotides, 5caC nucleotides, 5cxmC nucleotides, or a combination thereof.

In some embodiments, the oligonucleotide adapters do not comprise cytosine nucleotides in flow cell binding regions or primer binding sites in the oligonucleotide adapters.

In some embodiments, the classifier for detecting a cell proliferative disorder is further configured to determine a tissue of origin of the cell proliferative disorder.

In some embodiments, the classifier is trained using training vectors obtained from training biological samples, wherein a first subset of the training biological samples is identified as having a cell proliferative disorder, and a second subset of the training biological samples is identified as not having the cell proliferative disorder.

In another aspect, the present disclosure provides a method for sequencing a nucleic acid to provide hydroxymethylation state data of nucleic acid molecules in a biological sample, the method comprising:
a) obtaining a biological sample containing a nucleic acid;
b) ligating oligonucleotide adapters to at least a portion of the nucleic acids in the biological sample wherein the adapters comprise 5hmC nucleotides, 5gmC nucleotides, 5caC nucleotides, 5cxmC nucleotides, or a combination thereof, thereby generating ligated nucleic acids;
c) subjecting at least a portion of the ligated nucleic acids to conversion conditions necessary to convert unmethylated and methylated cytosines but not hydroxymethylated cytosines in the nucleic acids to uracil; and
d) sequencing the nucleic acids to obtain a nucleic acid sequence of the nucleic acids to provide hydroxymethylation state data in the nucleic acid molecules.

In some embodiments, the adapters comprise no cytosine nucleotides in flow cell binding regions or primer binding sites of the adapters.

In some embodiments, the method comprises after the ligation operation subjecting the ligated nucleic acids to glucosylation by β-GT/UDP-glucose to convert 5hmC nucleotides to 5gmC nucleotides.

In some embodiments, the conversion conditions comprise bisulfite treatment, enzymatic treatment, or a combination of both.

In some embodiments, the oligonucleotide adapters comprise all 5hmC nucleotides in place of cytosine nucleotides in a designed oligonucleotide adapter sequence.

In some embodiments, the oligonucleotide adapters comprise a mixture of 5gmC, 5caC, and/or 5cxmC nucleotides in place of cytosine nucleotides in a designed oligonucleotide adapter sequence.

In some embodiments, the enzymatic treatment comprises treatment with one or more of β-glucosyltransferase (β-GT), a cytosine dioxygenase enzyme (such as TET1, TET2, TET3, or functional variants thereof), carboxymethyltransferase, or AID/APOBEC.

In some embodiments, a sequence enrichment operation is performed after operation b) or prior to c).

In some embodiments, the sequence enrichment operation is a target capture hybridization.

In some embodiments, the ligated nucleic acids are amplified before sequencing.

In some embodiments, nucleic acid sequences obtained from sequencing are aligned to a reference genome.

In some embodiments, 5hmC-containing adapter oligonucleotides may be chemically synthesized using 5-hydroxymethyl modified cytidine phosphoramidites.

In some embodiments, adapter oligonucleotides containing a mixture of 5gmC and 5caC, may be produced by first synthesizing 5mC-containing adapters using phosphoramidite chemistry, and then enzymatically treating them with a TET enzyme plus β-GT/UDP-glucose.

A method for manufacturing oligonucleotide sequencing adapters, the method comprising:
a) synthesizing oligonucleotides containing 5mC by phosphoramidite chemistry;
b) converting the oligonucleotides with a TET enzyme plus β-GT/UDP-glucose under conditions sufficient to oxidize the oligonucleotide at the 5mC nucleotides; and
c) ligating the oxidized oligonucleotides to polynucleic acid molecules isolated from a biological sample.

In some embodiments, 5hmC-containing adapters may be directly synthesized using enzymatic oligonucleotide synthesis using terminal deoxynucleotidyl transferase (TdT) mediated enzymatic oligo synthesis.

In some embodiments, adapters containing a mixture of 5gmC and 5caC may be produced by first synthesizing 5mC-containing adapters using enzymatic oligonucleotide synthesis techniques and then enzymatically treating them with a TET enzyme plus β-GT/UDP-glucose.

In some embodiments, adapters containing 5mC may be produced by methylating adapters containing unmethylated cytosines using SAM-dependent C5-methyltransferase (C5-MT), or other DNA cytosine-5 methyltransferases.

A method for manufacturing oligonucleotide sequencing adapters, the method comprising:
a) synthesizing oligonucleotides containing 5gmC, 5caC, and/or 5cxmC by phosphoramidite chemistry; and
b) ligating the synthesized oligonucleotides to polynucleic acid molecules isolated from a biological sample.

In some embodiments, 5caC-containing adapters may be directly synthesized using enzymatic oligonucleotide synthesis techniques.

In another aspect, a method is provided for generating a hydroxymethylation profile for a biological sample obtained or derived from an individual, the method comprising:
a) obtaining a biological sample containing a nucleic acid;
b) ligating oligonucleotide adapters to the nucleic acids in the biological sample wherein the adapters comprise 5hmC, 5gmC, 5caC, 5cxmC, or a combination thereof and no cytosine nucleotides;
c) subjecting the ligated nucleic acids to conversion conditions necessary to convert unmethylated and methylated cytosines in the nucleic acids to uracil;
d) sequencing the nucleic acids to obtain a nucleic acid sequence of the nucleic acids, to provide hydroxymethylation state data in the nucleic acids; and
e) featurizing the hydroxymethylation state data and training a machine learning model to generate a methylation profile using the hydroxymethylation state data.

In some embodiments, the adapters comprise 5hmC, 5gmC, 5caC, 5cxmC, or a combination thereof and no cytosine nucleotides in flow cell binding regions or primer binding sites in the adapters

In some embodiments, the method comprises subjecting the ligated nucleic acids to glucosylation by β-GT/UDP-glucose to convert 5hmC to 5gmC, before subjecting to conversion conditions necessary to convert unmethylated and methylated cytosines in the nucleic acid to uracil.

In some embodiments, the nucleic acid sample is a cell-free DNA (cfDNA) sample.

In another aspect, the present disclosure provides a method for determining a hydroxymethylation profile of a cfDNA sample obtained or derived from an individual, the method comprising:
a) obtaining a biological sample containing a nucleic acid;
b) ligating oligonucleotide adapters to the nucleic acids in the biological sample wherein the adapters comprise 5hmC, 5gmC, 5caC, 5cxmC, or a combination thereof and no cytosine nucleotides;
c) subjecting the ligated nucleic acids to conversion conditions necessary to convert unmethylated and methylated cytosines in the biological sample's nucleic acids to uracil;
d) sequencing the nucleic acids to obtain a nucleic acid sequence of the nucleic acids, to provide hydroxymethylation state data in the nucleic acids; and
e) aligning the nucleic acid sequence of the converted nucleic acid molecules to a reference nucleic acid sequence to determine the hydroxymethylation profile of the individual.

In some embodiments, a nucleic acid sequencing library is prepared before the amplification.

In some embodiments, the adapters comprise 5hmC, 5gmC, 5caC, 5cxmC, or a combination thereof and no cytosine nucleotides in flow cell binding regions or primer binding sites in the adapters.

In some embodiments, the reference nucleic acid sequence is a reference genome.

In some embodiments, the method comprises subjecting the ligated nucleic acids to glucosylation by β-GT/UDP-glucose to convert 5hmC to 5gmC, before subjecting to conversion conditions necessary to convert unmethylated and methylated cytosines in the nucleic acid to uracil.

In some embodiments, the hydroxymethylation profile is associated with an abnormal cell state or disease and provides classification of a subject as having the abnormal cell state or disease

In some embodiments, the oligonucleotide adapters comprising a unique molecular identifier is ligated to unconverted nucleic acids in a cfDNA sample before a).

In some embodiments, the nucleic acid molecules are subjected to cytosine-to-uracil conversion conditions using chemical methods, enzymatic methods, or a combination thereof.

In some embodiments, the cfDNA in a biological sample is treated bisulfite, hydrogen sulfite, disulfite, or a combination thereof.

In some embodiments, the biological sample obtained from the subject contains nucleic acid molecules and is body fluids, stool, colonic effluent, urine, cerebrospinal fluid, blood plasma, blood serum, whole blood, isolated blood cells, cells isolated from the blood, or a combination thereof.

In some embodiments, the cell proliferative disorder is selected from stage 1 cancer, stage 2 cancer, stage 3 cancer, and stage 4 cancer.

In another aspect, a method is provided for generating a classifier for a nucleic acid sample obtained or derived from an individual, the method comprising:
a) obtaining a biological sample containing a nucleic acid;
b) ligating oligonucleotide adapters to the nucleic acids in the biological sample wherein the adapters comprise 5hmC, 5gmC, 5caC, 5cxmC, or a combination thereof and no cytosine nucleotides;
c) subjecting the ligated nucleic acids to conversion conditions necessary to convert unmethylated and methylated cytosines in the nucleic acids to uracil;
d) sequencing the nucleic acids to obtain a nucleic acid sequence of the nucleic acids, to provide hydroxymethylation state data in the nucleic acids; and
e) training a machine learning model to generate a classifier using the hydroxymethylation state data.

In some embodiments, the adapters comprise 5hmC, 5gmC, 5caC, 5cxmC, or a combination thereof and no cytosine nucleotides in flow cell binding regions or primer binding sites in the adapters.

In some embodiments, the method comprises subjecting the ligated nucleic acids to glucosylation by β-GT/UDP-glucose to convert hydroxymethylated C's to 5gmC, before subjecting to conversion conditions necessary to convert unmethylated and methylated cytosines in the nucleic acid to uracil.

In another aspect, the present disclosure provides a method for detecting a cell proliferative disorder in a subject, the method comprising:
a) obtaining a biological sample containing a nucleic acid;
b) ligating oligonucleotide adapters to the nucleic acids in the biological sample wherein the adapters comprise 5hmC, 5gmC, 5caC, 5cxmC, or a combination thereof and no cytosine nucleotides;
c) subjecting the ligated nucleic acids to conversion conditions necessary to convert unmethylated and methylated cytosines in the nucleic acids to uracil;
d) sequencing the nucleic acids to obtain a nucleic acid sequence of the nucleic acids, to provide hydroxymethylation state data in the nucleic acids; and
f) processing the hydroxymethylation state data using a machine learning model trained to be capable of distinguishing between healthy subjects and subjects with a cell proliferative disorder to provide an output value associated with presence of a cell proliferative disorder, thereby indicating the presence of a cell proliferative disorder in the subject.

In some embodiments, the adapters comprise 5hmC, 5gmC, 5caC, 5cxmC, or a combination thereof and no cytosine nucleotides in flow cell binding regions or primer binding sites in the adapters.

In some embodiments, the method comprises subjecting the ligated nucleic acids to glucosylation by β-GT/UDP-glucose to convert hydroxymethylated C's to 5gmC, before subjecting to conversion conditions necessary to convert unmethylated and methylated cytosines in the nucleic acid to uracil.

In various embodiments, the different types of cell proliferative disorders are selected from colorectal cancer, breast cancer, ovarian cancer, prostate cancer, lung cancer, pancreatic cancer, uterine cancer, liver cancer, esophagus cancer, stomach cancer, thyroid cancer, or bladder cancer,

In some embodiments, the machine learning classifier is tailored to provide pre-selected sensitivity and specificity for the different types of cell proliferative disorder to be detected depending on needs of cancer diagnosis and confirmatory diagnosis for a cell proliferative disorder that is colorectal cancer, breast cancer, ovarian cancer, prostate cancer, lung cancer, pancreatic cancer, uterine cancer, liver cancer, esophagus cancer, stomach cancer, thyroid cancer, or bladder cancer, or a combination thereof.

In some embodiments, the machine learning model classifies the presence or susceptibility of the cancer at a sensitivity of at least about 80%. In some embodiments, the machine learning model classifies the presence or susceptibility of the cancer at a sensitivity of at least about 90%. In some embodiments, the machine learning model classifies the presence or susceptibility of the cancer at a sensitivity of at least about 95%. In some embodiments, the machine learning model classifies the presence or susceptibility of the cancer at a positive predictive value (PPV) of at least about 70%. In some embodiments, machine learning model classifies the presence or susceptibility of the cancer at a PPV of at least about 80%. In some embodiments, the machine learning model classifies the presence or susceptibility of the cancer at a PPV of at least about 90%. In some embodiments, the machine learning model classifies the presence or susceptibility of the cancer at a PPV of at least about 95%. In some embodiments, machine learning model classifies the presence or susceptibility of the cancer at a PPV of at least about 99%. In some embodiments, the machine learning model classifies the presence or susceptibility of the cancer at a negative predictive value (NPV) of at least about 80%. In some embodiments, the machine learning model classifies the presence or susceptibility of the cancer at a NPV of at least about 90%. In some embodiments, the machine learning model classifies the presence or susceptibility of the cancer at a NPV of at least about 95%. In some embodiments, the machine learning model classifies the presence or susceptibility of the cancer at a NPV of at least about 99%. In some embodiments, the machine learning model classifies the presence or susceptibility of the cancer of the subject with an Area Under Curve (AUC) of at least about 0.90. In some embodiments, the machine learning model classifies the presence or susceptibility of the cancer of the subject with an AUC of at least about 0.95. In some embodiments, the machine learning model classifies the presence or susceptibility of the cancer of the subject with an AUC of at least about 0.99.

In some embodiments, the conversion conditions comprise bisulfite treatment, enzymatic treatment, or a combination of both.

In some embodiments, the oligonucleotide adapters comprise all 5hmC nucleotides in place of cytosine nucleotides in flow cell binding regions and optionally also primer binding sites in the adapters in a pre-determined oligonucleotide adapter sequence.

In some embodiments, the oligonucleotide adapters comprise a mixture of 5gmC and 5caC or 5cxmC and cytosine nucleotides in a designed oligonucleotide adapter sequence.

In some embodiments, the enzymatic treatment comprises treatment with one or more of β-glucosyltransferase (β-GT), a cytosine dioxygenase enzyme (such as TET1, TET2, TET3, or functional variants thereof), carboxymethyltransferase, or AID/APOBEC.

In some embodiments, the enzymatic treatment use of TET enzymes occurs to the adapters prior to ligation.

In some embodiments, a sequence enrichment operation is performed after operation b) or prior to c).

In some embodiments, the sequence enrichment operation is a target capture hybridization.

In some embodiments, the ligated nucleic acids are amplified before sequencing.

In some embodiments, nucleic acid sequences obtained from sequencing are aligned to a reference genome.

In some embodiments, the hydroxymethylation state data is featurized and processed using a trained machine learning model that is trained to classify the sample into groups according to predesignated or preselected biological properties.

In some embodiments, a set of features are identified from the nucleic acid sequences to be processed using a machine learning model. The set of features can correspond to properties of the nucleic acid sequences in the biological sample.

In some embodiments, the properties of the nucleic acid sequences are selected from the presence or absence of pre-cancer, cancer or a stage of cancer, or a prognosis of cancer in an individual from whom the sample was obtained.

In another aspect, the present disclosure provides a method for monitoring minimal residual disease in a subject previously treated for disease comprising:
determining a hydroxymethylation profile as described herein as a baseline hydroxymethylation state and repeating an analysis to determine the hydroxymethylation profile at one or more predetermined time points wherein a change from baseline indicates a change in the minimal residual disease status at baseline in the subject.

In some embodiments, the minimal residual disease is selected from response to treatment, tumor load, residual tumor post-surgery, relapse, secondary screen, primary screen, and cancer progression.

In another aspect, a method is provided for determining response to treatment.

In another aspect, a method is provided for monitoring tumor load.

In another aspect, a method is provided for detecting residual tumor post-surgery.

In another aspect, a method is provided for detecting relapse.

In another aspect, a method is provided for use as a secondary screen.

In another aspect, a method is provided for use as a primary screen.

In another aspect, a method is provided for monitoring cancer progression.

In an aspect, the present disclosure provides a system comprising a machine learning model classifier for detecting a cell proliferative disorder, the system comprising:
a) a computer-readable medium comprising a classifier operable to classify subjects as having the cell proliferative disorder or not having the cell proliferative disorder based on hydroxymethylation state data obtained from a nucleic acid library generated using oligonucleotide adapters to the nucleic acids in the biological sample wherein the adapters comprise 5hmC, 5gmC, 5caC, 5cxmC, or a combination thereof and no cytosine nucleotides; and
b) one or more processors for executing instructions stored on the computer-readable medium.

In some embodiments, the adapters comprise 5hmC, 5gmC, 5caC, 5cxmC, or a combination thereof and no cytosine nucleotides in flow cell binding regions or primer binding sites in the adapters.

In some embodiments, the machine learning model classifier for detecting a cell proliferative disorder comprises tissue of origin determination.

In some embodiments, the system comprises the classifier loaded into a memory of a computer system, the machine learning model trained using training vectors obtained from training biological samples, a first subset of the training biological samples identified as having a cell proliferative disorder and a second subset of the training biological samples identified as not having a cell proliferative disorder.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Examples of the present disclosure will now be described, by way of example only, with reference to the attached Figures. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A and FIG. 1B provide schematics showing example adapters (FIG. 1A) and methods of use thereof (FIG. 1B). FIG. 1A provides a generalized example of adapters used in hydroxymethylation sequencing. Adapters can contain any of the following modified cytosines in flow cell and primer binding regions: 5hmC, 5gmC, 5caC, or 5cxmC. Cytosines in UMI regions can be unmodified or modified with 5mC, 5hmC, 5gmC, 5caC, or 5cxmC. 5m (5-methyl), 5hm (5-hydroxymethyl), 5gm (β-glucosyl-5-hydroxymethyl), 5ca (5-carboxyl), 5cxm (5-carboxymethyl), UMI (unique molecular barcode). FIG. 1B provides examples of processes to generate adapters for hydroxymethylation sequencing. Adapters can be designed and synthesized using (i) mC nucleotides or (ii) a combination of 5hmC, 5gmC, 5caC, or 5cxmC nucleotides at positions requiring protection from deamination. For process (i), synthesized adapters may be oxidized and optionally (*) glucosylated before use in ligation. For process (ii), adapters are ready for use in ligation. C (cytosine), m (methyl), 5hm (5-hydroxymethyl), 5gm (β-glucosyl-5-hydroxymethyl), 5ca (5-carboxyl), 5cxm (5-carboxymethyl).

FIG. 2 provides a schematic of an example 5hmC-seq assay overview. Operations of the 5hmC-seq assay start with adapters that have been protected against downstream enzymatic conversion. The target enrichment operation is optional (*).

FIG. 3 provides a schematic of a computer system that is programmed or otherwise configured with the machine learning models and classifiers in order to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure relates generally to oligonucleotide adapter compositions useful for cytosine hydroxymethylation status sequencing of nucleic acids in a biological sample. DNA methylation at the 5-carbon position of cytosine (5-methylcytosine; 5mC) is an epigenetic mark with functional roles in gene silencing, nucleosome positioning, and chromatin organization. In humans, DNA methylation occurs predominantly at cytosines in CpG dinucleotides. Methylation marks are heritable, and their genome-wide profiles differ from tissue to tissue. In cancer, gene-specific methylation profiles become aberrant but retain similarity to the tissue of origin. These properties make methylation marks highly useful biomarkers for cancer diagnosis and prognosis.

Circulating cell-free DNA (cfDNA) is released into blood from dying apoptotic or necrotic cells, and hence represents a snapshot of cell death across the entire human body. In tumors, some fraction of cells continually dies and releases DNA into circulation as cell-free tumor-derived DNA (ctDNA) fragments. Knowledge of tumor-specific DNA methylation patterns can be harnessed as a methylation atlas to examine cfDNA and to determine whether a given fragment thereof originated from a tumor or normal cell type.

Hydroxymethylation is another epigenetic modification at the 5-carbon position of cytosine (5hmC). This modification may be involved in active demethylation and may play a role in regulating gene expression. In active demethylation pathways, 5hmC may be generated as the first operation in the iterative oxidation of 5mC. Investigations into the genome-wide distribution of 5hmC have demonstrated a dynamic landscape that strongly associates with gene expression. Alterations in 5hmC profiles may be associated with a wide range of disease states including cell proliferative disorders.

The term “cell proliferative disorder”, as used herein, may generally refer to a disorder or disease that comprises disordered or aberrant proliferation of cells. In some non-limiting examples, the disorder is colorectal cell proliferation, prostate cell proliferation, lung cell proliferation, breast cell proliferation, pancreatic cell proliferation, ovarian cell proliferation, uterine cell proliferation, liver cell proliferation, esophagus cell proliferation, stomach cell proliferation, or thyroid cell proliferation. In some embodiments, the cell proliferative disorder is colon adenocarcinoma, liver hepatocellular carcinoma, lung adenocarcinoma, lung squamous cell carcinoma, ovarian serious cystadenocarcinoma, pancreatic adenocarcinoma, prostate adenocarcinoma, or rectum adenocarcinoma. The term “normal” or “healthy”, as used herein, may generally refer to a cell, tissue, plasma, blood, biological sample, or subject not having a cell proliferative disorder.

Improvements in library preparation that capture improved quality hydroxymethylation information of nucleic acids in a biological sample may be necessary to increase the sensitivity of classification models and associated clinical screening methods.

### I. Library Preparation and Adapter Ligation for Enzymatic Hydroxymethylation Sequencing

Methods are provided for the preparation of a sequencing library for detecting 5hmC, 5-formylcytosine (5fC), and 5caC in a nucleic acid molecule from a biological sample. These methods may provide improved library yield and quality that is scalable, more manageable, and provides improved adapter protection over other hydroxymethylation sequencing approaches. These methods may also provide base-resolution 5hmC data in short-read sequencing that is more cost-effective and less error prone than long-read sequencing approaches.

The methods described herein provide a library that is acceptable for DNA hydroxymethylation sequencing applications, but also non-methylation sequencing applications, thereby providing sequencing data for multiple applications from a single sample. The resulting raw sequencing data may be used for hydroxymethylation state analysis, as well as more conventional cfDNA analysis, such as copy number alterations, germline variant detection, somatic variant detection, nucleosome positioning, transcription factor profiling, chromatin immunoprecipitation, and the like.

### A. Adapter Ligation for Sequencing Applications

In one aspect, the present methods may preserve the integrity and information of nucleic acid sequences for hydroxymethylation profiling. In one example, combining dsDNA adapter ligation before 5hmC protection and APOBEC conversion (e.g., deamination) may preserve fragment endpoint information while providing the highest possible library complexity for library preparation, thereby providing greater sensitivity to detect rare events, such as hydroxymethylated ctDNA. This method may be applied to either sample target enrichment or directly for genome-wide sequencing.

Performing adapter ligation prior to 5hmC protection and APOBEC conversion of a sample nucleic acid may allow for implementation of dsDNA-dependent adapter ligation methods, which maintain endpoint information while producing high complexity libraries. In addition, when the fragment length of the sample nucleic acid is small, such as with cfDNA (modal size=167 base pairs, bp), adapter ligation may extend the length of the DNA by approximately twice the length of the adapters (due to a double-sided ligation), which provides an advantage over unligated cfDNA due to significantly increased recovery efficiency during solid phase reversible immobilization (SPRI)-bead based reaction cleanup operations. Preserving endpoint information of a nucleic acid sequence in the biological sample may allow for more accurate analysis of fragmentation patterns in cfDNA, which can be used as a feature in machine learning models. In order to ligate adapter oligonucleotides prior to a protection/conversion workflow process, the cytosines in an oligonucleotide adapter that bind to a flow cell surface or a sequencing primer binding site are first modified or protected from deamination that occurs during a conversion operation because a C-to-T substitution during conversion may obstruct sequencing. In some embodiments, this approach may reduce or eliminate the limitations of TAB-seq and ACE-seq by using adapters containing 5hmC, or a mixture of 5gmC and 5caC, in sequence positions where cytosine would normally be positioned during adapter design for flow cell attachment and sequencing primer binding. These methods, unlike 5hmC-Seal combined with long-read sequencing, use short-read sequencing, which, in some embodiments, may be more amenable to the applications discussed herein.

In some embodiments, 5hmC-containing adapter oligonucleotides may be directly synthesized using 5-hmC phosphoramidites. After ligation of 5hmC-containing adapters to cfDNA, the 5hmC nucleotides in the adapter oligonucleotide, as well as the sample nucleic acid library insert, may be subjected to glucosylation using β-glucosyltransferase (β-GT) and the substrate, UDP-glucose, during a labeling operation of hydroxymethylated cytosines. Glucosylation of hydroxymethylated cytosines in sample nucleic acids may protect the modified cytosines from deamination by subsequent treatment, for example, with bisulfite or APOBEC enzyme.

In some embodiment, oligonucleotide adapters containing a mixture of 5gmC and 5caC, may be produced by first synthesizing 5mC-containing adapters using phosphoramidite chemistry, and then enzymatically treating them with a TET enzyme plus β-GT/UDP-glucose. Chemical synthesis of adapters containing 5mC may be both more efficient with less early truncation products and less expensive than that of 5hmC-containing adapters.

In some embodiments, 5hmC-containing adapters may be produced using enzymatic oligonucleotide synthesis techniques. In some embodiments, enzymatic oligonucleotide synthesis methods employ terminal deoxynucleotidyl transferase (TdT), a template independent polymerase that attaches supplied deoxynucleotides to 3′-OH ends of DNA.

In one example, oligonucleotide adapters may be ligated to the 5′ and 3′ ends of a population of nucleic acid fragments in a biological sample to produce a sequencing library. In one example, a collection of nucleic acid adapters is ligated to the nucleic acid fragments in a sample where the collection of adapters includes equal parts of 4 bp, 5 bp, and 6 bp unique molecular identifier (UMI) sequences followed by an invariant thymidine (T) at the last position (e.g., the 3′ end) to enable T/A overhang ligation. Thus, the UMIs may be located adjacent to the library insert nucleic acid. During sequencing, the UMIs may also be sequenced as a part of the read at the 5′ end (alternatively, the UMIs may be in line with the library insert at the sequencing read level). The invariant T may be staggered over 3 positions to maintain base diversity at the sequenced position. In contrast, using a single-length UMI with an invariant thymidine may lead to low-complexity sequencing at the position corresponding to the invariant thymidine resulting in reduced sequencing quality. The first 4 bp of each UMI together comprise a set of 4-bp core UMI sequences that have an edit distance of greater than or equal to 2 and are nucleotide and color balanced. Using a single length core UMI, despite variable-length UMI sequences, may facilitate the use of bioinformatic tools that are built for single-length UMIs for UMI extraction and deduplication. Thus, the 4-bp core sequence may serve as a recognition sequence that informs the bioinformatic tool to trim 5, 6, or 7 bases (inclusive of the invariant T), thereby maintaining precise cfDNA end point information. The use of UMIs may permit read deduplication, single-stranded error correction, and duplex reconstruction after sequencing, thereby permitting use of a read's reverse complement to enhance error correction, also referred to as double-stranded error correction. In another example, unique dual indexes (UDI) are additional sequences that may be added to the UMI-containing adapters during library preparation to provide sample barcoding and de-multiplexing of samples after sequencing. In various examples, the UDI sequences are 4 bp, 5 bp, 6 bp, 7 bp, 8 bp, or 12 bp in length.

In various embodiments, the oligonucleotide adapters may include UMIs of 4 bp to 6 bp in length with a 5′ thymidine overhang. The UMIs are designed to be non-unique (e.g., drawn from a specific, constrained set of sequences).

In some embodiments, some UMIs contain one or more methylcytosine bases. The efficiency of the enzymatic methylation conversion reactions (including TET oxidation and APOBEC deamination) can be assessed based on the fraction of UMIs that do not match the specific, constrained set of designed UMI sequences by a UMI mismatch rate. The UMI mismatch rate may be used as an embedded quality control metric to assess sequencing library quality. In addition, if perfect UMI matches are required in the bioinformatics pipeline, then the UMI mismatch rate may be used as a filter to remove individual reads that may be of lower quality due to incomplete conversion.

In various embodiments, the UMI mismatch rate is less than 6%, less than 5%, less than 4%, less than 3%, or less than 2%.

In some embodiments, the UMIs contain one or more cytosines containing modifications that may be used to monitor the enzymatic activities. Non-limiting examples of these modified bases include 5mC, 5hmC, 5fC, and 5cxmC.

In some examples, the cytosines present in adapter nucleic acid are modified with a 5-methyl group or 5-hydroxymethyl group to prevent C-to-T conversion in the adapters.

In one example, the cytosines present in adapter nucleic acid are modified with a 5hmC, 5gmC, 5caC, or 5cxmC group to prevent cytosine (C)-to-uracil (U) conversion in the adapters.

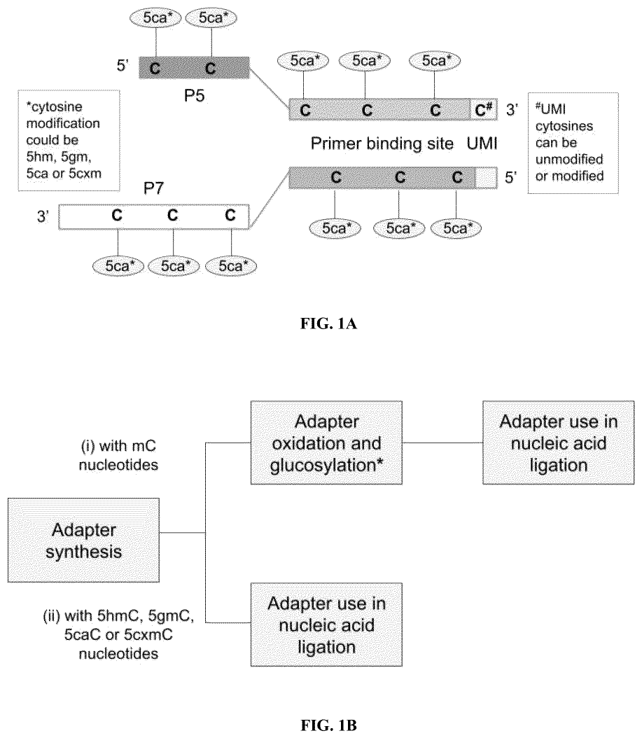

FIG. 1A provides a generalized example of adapters used in hydroxymethylation sequencing. Adapters can contain any of the following modified cytosines in flow cell and primer binding regions: 5hmC, 5gmC, 5caC, or 5cxmC. Cytosines in UMI regions can be unmodified or modified with 5mC, 5hmC, 5gmC, 5caC, or 5cxmC. 5m (5-methyl), 5hm (5-hydroxymethyl), 5gm (β-glucosyl-5-hydroxymethyl), 5ca (5-carboxyl), 5cxm (5-carboxymethyl), UMI (unique molecular barcode).

FIG. 1B provides examples of processes to generate adapters for hydroxymethylation sequencing. Adapters can be designed and synthesized using (i) mC nucleotides or (ii) a combination of 5hmC, 5gmC, 5caC, or 5cxmC nucleotides at positions requiring protection from deamination. For process (i), synthesized adapters may be oxidized and optionally (*) glucosylated before use in ligation. For process (ii), adapters are ready for use in ligation. C (cytosine), m (methyl), 5hm (5-hydroxymethyl), 5gm (β-glucosyl-5-hydroxymethyl or 5-(β-glucosyloxymethyl)cytosine), 5ca (5-carboxyl), 5cxm (5-carboxymethyl).

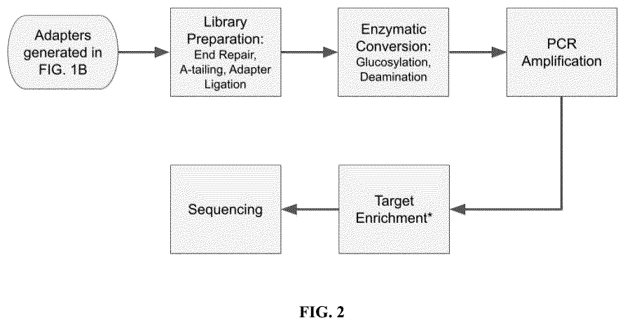

FIG. 2 provides a schematic of an example 5hmC-seq assay overview. Operations of the 5hmC-seq assay start with adapters, e.g., generated from FIG. 1B that have been protected against downstream enzymatic conversion. The target enrichment operation is optional (*).

One advantage of this approach may be that adapter ligation before conversion maintains fragment endpoint and length information as compared to an approach that performs bisulfite conversion followed by ssDNA adapter ligation. The considerable degradation of nucleic acid before ligating adapters may result in loss of informative fragment endpoint and length information.

Enzymatic (e.g., using APOBEC) conversion of C-to-U may be less degradative on sample nucleic acid fragments and may result in more complete and uniform coverage as compared to bisulfite conversion methods. Bisulfite degradation of DNA may not be uniform, so some sequences may be preferentially degraded over others, including CG dinucleotides, which are the very sites being interrogated in hydroxymethylation sequencing. Thus, the enzymatic approach may provide a higher coverage of CpG sites than bisulfite conversion methods using the same number of unique reads, and greater uniformity of captured reads in target enrichment applications. Furthermore, non-bisulfite methods (e.g., enzymatic conversion) may provide increased resolution of biological signal, and specifically, the ability to differentiate 5mC and 5hmC in a nucleic acid sequence. This information and additional resolution may be informative in computational approaches and other methods.

In some examples, subjecting the DNA or the barcoded DNA to enzymatic reactions that convert unmodified, methylated and hydroxymethylated cytosine nucleobases of the sample DNA or the barcoded DNA into uracil nucleobases includes performing enzymatic conversion.

In various examples, glucosylation of 5hmC in nucleic acids from a biological sample protects the 5hmC from deamination. Deaminases may be used to convert unmodified C, 5mC, and 5hmC to U or a derivative thereof. Non-limiting examples of deaminases include APOBEC (apolipoprotein B mRNA editing enzyme, catalytic polypeptide-like). Embodiments described herein utilize APOBEC in sufficient quantities to overcome sequence bias in deamination of unmethylated or methylated cytosine. Moreover, embodiments involving APOBEC conversion rather than bisulfite conversion may provide substantially less damage to the nucleic acids from a biological sample.

In some examples, a 5hmC sequencing method may include: contacting an aliquot of the nucleic acid sample with β-GT in the absence of a TET dioxygenase, followed by treatment with cytidine deaminase (e.g., an APOBEC) to produce a reaction product in which substantially all the 5hmCs in the aliquot are glucosylated, and substantially all the unmodified cytosines and 5mCs are converted to uracils. After PCR amplification, the uracils are substituted with thymidines, and thus, cytosine and 5mC become indistinguishable when sequenced. The resultant reaction product can be sequenced and compared to a reference sequence to differentiate 5hmCs from cytosines and from 5mCs. Differentiation of these moieties may allow mapping of these modified nucleotides to a reference sequence. A reference nucleic acid sequence may be obtained by sequencing a nucleic acid sample that is not reacted with any β-GT or deaminase. Alternatively, a reference sequence may be used for mapping where the reference sequence is a known reference nucleic acid sequence (e.g., obtained from a database of sequences or a reference genome).

### B. 5hmC Nucleic Acid Sequencing

Several sequencing methods may be used to identify 5hmC, including Tet-assisted bisulfite sequencing (TAB-seq), 5hmC selective chemical labeling technique (e.g., 5hmC-seal), APOBEC-coupled epigenetic sequencing (ACE-seq), and DNA immunoprecipitation-coupled chemical-modification assisted bisulfite sequencing (DIP-CAB-seq). Each method may have advantages and disadvantages.

In TAB-seq, 5hmC nucleotides are protected by modification to 5-(β-glucosyloxymethyl)cytosine (5gmC) using T4 β-glucosyltransferase (β-GT), and 5mC bases are converted to 5caC using mTet1. Subsequently, all C and 5caC nucleotides may be deaminated by bisulfite conversion to U or 5caU, respectively. However, bisulfite may degrade 90-99% of DNA, so while TAB-seq achieves single base 5hmC resolution, TAB-seq may require relatively large amounts of DNA to mitigate bisulfite-mediated degradation. Hence, the high DNA mass requirements may prevent TAB-seq from being adopted to sequence 5hmC in cfDNA samples, which may be a limited analyte.

In 5hmC-Seal, β-GT is used to label 5hmC with an azide-modified glucose (UDP-6-N3-Glu), and the azide group allows subsequent covalent attachment of biotin via click chemistry. Streptavidin beads are used to affinity capture biotin-5gmC containing DNA fragments while unbound fragments are washed away. Captured DNA fragments are then PCR amplified and sequenced. This technique does not include operations that allow disambiguation of 5hmC from other modified/unmodified C bases using short-read sequencing methods (e.g., 5gmC reads out as C). As a result, the method may only identify cfDNA fragments which contain at least one 5hmC, but the number and specific positions of the 5hmC are unknown. The long-read sequencing technology SMRT sequencing can be used to obtain single nucleotide resolution of 5hmC from 5hmC-Seal captured DNA fragments. Short-read sequencing may be preferred over long-read sequencing, which is more cost-effective and less error prone.

Like TAB-seq, ACE-seq employs β-GT to protect 5hmC with a glucose moiety. Unlike TAB-seq, the conversion/deamination operation in ACE-seq is enzymatically mediated by APOBEC instead of chemically by bisulfite. Thus, ACE-seq can require less input DNA than TAB-seq, but the method may still have disadvantages. First, the cfDNA input volume may be very low, e.g., only about 4 μL (estimated from the difference between the total volume of the glucosylation reaction that is about 5 μL and the total volume of the substrate, enzyme, and concentrated buffer components that is about 1 μL). cfDNA samples are generally in the low hundreds of picogram (pg)/μL range (e.g., ˜200 pg/μL); hence, the method may only support low cfDNA mass inputs (<1-2 ng) without devising a workaround for concentrating cfDNA. Hence, this low cfDNA input volume may inherently limit the sensitivity of the method for identifying very rare 5hmC in cfDNA as biomarkers in disease applications. Second, enzymatic glucosylation and deamination of cfDNA is carried out before adapter ligation in ACE-seq. Generally, a dsDNA-dependent adapter ligation is the first operation in an NGS application. However, if adapter ligation is carried out before deamination, then the Cs in the adapters would deaminate to U, which would not be compatible with Illumina platform sequencing applications. By deaminating cfDNA before ligation, the adapter cytosines may remain unaltered. However, the C-to-U conversion in the cfDNA insert from the deamination may produce non-complementary strands. Thus, adapter ligation strategies after deamination of cfDNA may require unconventional ssDNA-based ligation approaches. In ACE-seq, ssDNA-based ligation may be accomplished by employing the Accel Methyl-NGS kit (Swift Biosciences) to introduce Illumina adapter sequences. This particular ssDNA ligation method, however, may add an unknown number of low complexity bases to the 3′ ends of ssDNA (to serve as a primer binding site for second strand synthesis), and thus, may erase 3′ end point information. Additionally, requiring ssDNA-based ligation may negate the possibility of detecting a given read's reverse complement strand (because the cfDNA is denatured before ligation) using duplex UMI strategies. Thus, ssDNA-based libraries may lose reverse complement strand information, which allows for greater sequencing error suppression.

If the test converted nucleic acid sequence is a T that corresponds to the reference C at a specified CpG locus, then the C was unmethylated in the original test nucleic acid fragment. In contrast, if the test converted nucleic acid sequence and the reference sequence are both C at a specified CpG locus, then the C was hydroxymethylated in the original test nucleic acid fragment.

In some examples, the nucleic acid sequence of the converted nucleic acid molecules is sequenced at a depth of between about 50-500×, about 25-1000×, about 50-500×, about 250-750×, about 500-200×, about 750-1500×, or about 100-2000×. In some embodiments, a nucleic acid sequence is sequenced at a depth of greater than 100× or greater than 500×.

In some examples, the nucleic acid sequence of the converted nucleic acid molecules is sequenced at a depth of about 500×, about 1000×, about 2000×, about 3000×, about 4000×, about 5000×, about 6000×, about 7000×, about 8000×, about 9000×, about 10000×, or greater than 5000×.

In some examples, the nucleic acid sequence of the converted nucleic acid molecules is sequenced at a depth of about 300× unique, about 400× unique, about 500× unique, about 600× unique, about 700× unique, about 800× unique, about 900× unique, or about 1000× unique, or greater than 500× unique.

### C. Hydroxymethylation Profiling

In various examples, when enzymatic hydroxymethylation sequencing is complete, assays may be used to analyze the hydroxymethylation state of nucleic acids in a biological sample. In some examples, whole genome enzymatic hydroxymethyl sequencing (“WG EHM-seq”) provides high resolution sequencing by characterizing DNA hydroxymethylation state of nearly every cytidine nucleotide in the genome. Other targeted methods, such as targeted enzymatic hydroxymethyl sequencing (“TEHM-seq”), may be useful for methylation analysis.

The hydroxymethylation profile of cfDNA can be identified by applying sequence alignment methods to map hydroxymethyl sequencing reads from whole genome or targeted hydroxymethyl sequencing of a human reference genome. Non-limiting examples of sequence alignment methods include bwa-meth, bismark, Last, GSNAP, BSMAP, NovoAlign, Bison, Metagenomic Phylogenetic Analysis (for example, MetaPhlAn2), BLAT, Burrows-Wheeler Aligner (BWA), Bowtie, Bowtie2, Bfast, BioScope, CLC bio, Cloudburst, Eland/Eland2, GenomeMapper, GnuMap, Karma, MAQ, MOM, Mosaik, MrFAST/MrsFAST, PASS, PerM, RazerS, RMAP, SSAHA2, Segemehl, SeqMap, SHRIMP, Slider/SliderII, Srprism, Stampy, vmatch, ZOOM, and the SOAP/SOAP alignment tool.

The use of duplex-UMIs in hydroxymethyl sequencing may increase the accuracy of determining a true hydroxymethylation state of a nucleic acid molecule. This method can account for possible errors introduced during, for example, extraction (DNA damage), library preparation (end repair fill-in), enzymatic conversion (underconversion or overconversion), PCR (base-incorporation errors), and sequencing (base-calling errors). Increasing accuracy of hydroxymethylation state determination may improve featurization and classifier generation for stratifying a population using these hydroxymethylation-based epigenetic sequence differences. This method does not rely on an index barcode for error correction.

D. Combination with Nucleic Acid Enrichment Methods

In another aspect, the methods comprise enrichment for desired nucleic acids. In some embodiments, the present hydroxymethyl sequencing methods may be performed on samples of nucleic acids that are enriched for desired nucleic acid sequences. In some embodiments, the present hydroxymethyl sequencing methods comprise a nucleic acid enrichment operation. In some embodiments, nucleic acid enrichment methods may be combined with a method for sequencing hydroxymethylated cell-free DNA. In some embodiments, the method comprises adding an affinity tag to only hydroxymethylated DNA molecules in a sample of cfDNA, enriching for the DNA molecules that are tagged with the affinity tag, and sequencing the enriched DNA molecules. In some embodiments, complementary nucleic acid molecules are used in enrichment methods to target genomic sequences with methylation statuses that are implicated in cancer progression, detection, prognosis, or treatment response.

In some embodiments, the nucleic acids are predetermined by size, nucleobase content, or nucleic acid sequence. Certain enrichment methods may be applied in combination with the methods described herein such as U.S. Patent Publication No. US20200123616 and International Patent Publication No. WO2017176630A1, each of which is incorporated by reference herein.

The terms “enrich” and “enrichment” refers to a partial purification of analytes that have a certain feature (e.g., nucleic acids that contain hydroxymethylcytosine) from analytes that do not have the feature (e.g., nucleic acids that do not contain hydroxymethylcytosine).

Enrichment may increase the concentration of the analytes that have the feature (e.g., nucleic acids that contain hydroxymethylcytosine) by at least 2-fold, at least 5-fold, or at least 10-fold relative to the analytes that do not have the feature. After enrichment, at least 10%, at least 20%, at least 50%, at least 80%, or at least 90% of the analytes in a sample may have the feature used for enrichment. For example, at least 10%, at least 20%, at least 50%, at least 80%, or at least 90% of the nucleic acid molecules in an enriched composition may contain a strand having one or more hydroxymethylcytosines that have been modified to contain a capture tag. Other definitions of terms may appear throughout the specification.

The enrichment operation of the method may be done using magnetic streptavidin beads, although other supports may be used. As noted above, the enriched cfDNA molecules (which correspond to the hydroxymethylated cfDNA molecules) may be amplified by PCR and then sequenced. In such embodiments, the enriched cfDNA sample may be amplified using one or more primers that hybridize to the added adapters (or complements thereof). In some embodiments, the enriched DNA sample is deaminated, e.g., using an APOBEC, prior to PCR amplification. This sequence of operations may allow base-resolution determination of 5hmC modifications on the enriched DNA.

In some embodiments, the deaminated enriched DNA may be amplified using one or more primers that hybridize to Y-shaped adapters. In embodiments in which Y-shaped adapters (Y-adapters) are added, the adapter-ligated nucleic acids may be amplified by PCR using two primers: a first primer that hybridizes to the single-stranded region of the top strand of the adapters, and a second primer that hybridizes to the complement of the single-stranded region of the bottom strand of the Y-adapters (or hairpin adapters, after cleavage of the loop). For example, in some embodiments the Y-adapters used may have P5 and P7 arms (which sequences are compatible with Illumina's sequencing platform) and the amplification products may have the P5 sequence at one and the P7 sequence at the other. These amplification products can be hybridized to an Illumina sequencing substrate and sequenced. In some embodiments, the pair of primers used for amplification may have 3′ ends that hybridize to the Y-adapters and 5′ tails that either have the P5 sequence or the P7 sequence. In these embodiment, the amplification products may also have the P5 sequence at one and the P7 sequence at the other. These amplification products can be hybridized to an Illumina sequencing substrate and sequenced. This amplification operation may be done by limited cycle PCR (e.g., 5-20 cycles).

A method that comprises (a) obtaining a sample comprising circulating cell-free DNA, (b) enriching for the hydroxymethylated DNA in the sample and (c) independently quantifying the amount of nucleic acids in the enriched hydroxymethylated DNA that map to (e.g., have sequences that correspond to) each of one or more target loci (e.g., at least 1, at least 2, at least 3, at least 4, at least 5, or at least 10 target loci) is also provided. This method may further comprise: (d) determining whether one or more nucleic acid sequences in the enriched hydroxymethylated DNA are over-represented or underrepresented in the enriched hydroxymethylated DNA, relative to a control. The identity of the nucleic acids that are over-represented or underrepresented in the enriched hydroxymethylated DNA (and, in certain cases the extent to those nucleic acids are overrepresented or underrepresented in the enriched hydroxymethylated DNA) can be used to make a diagnosis, a treatment decision or a prognosis. For example, in some cases, analysis of the enriched hydroxymethylated DNA may identify a signature that correlates with a phenotype, as discussed above. In some embodiments, the amount of nucleic acid molecules in the enriched hydroxymethylated DNA that map to each of one or more target loci (e.g., the genes/intervals listed below) may be quantified by qPCR, digital PCR, arrays, sequencing, or any other quantitative method.

In some embodiments, the method may comprise attaching labels to DNA molecules that comprise one or more hydroxymethylcytosine and methylcytosine nucleotides in a sample of cfDNA, wherein the hydroxymethylcytosine nucleotides are labeled with a first capture tag and the methylcytosine nucleotides are labeled with a second capture tag that is different to the first capture, to produce a labeled sample; enriching for the DNA molecules that are labeled; and sequencing the enriched DNA molecules. This embodiment of the method may comprise separately enriching the DNA molecules that comprise one or more hydroxymethylcytosines and the DNA molecules that comprise one or more methylcytosine nucleotides. The labeling may be adapted from the methods described above or from Song et al. (“Simultaneous single-molecule epigenetic imaging of DNA methylation and hydroxymethylation”, Proc. Natl. Acad. Sci. 2016 113: 4338-43, which is incorporated by reference herein), where capture tags are used instead of fluorescent labels.

In some embodiments, the enrichment methods may be implemented by ligating the DNA is to a universal adapters, e.g., an adapters that ligates to both ends of the fragments of cfDNA. In certain cases, the universal adapters may be done by ligating a Y-adapters (or hairpin adapters) onto the ends of the cfDNA, thereby producing a double stranded DNA molecule that has a top strand that contains a 5′ tag sequence that is not the same as or complementary to the tag sequence added the 3′ end of the strand. The DNA fragments used in the initial operation of the method may be non-amplified DNA that has not been denatured beforehand. As shown in FIG. 1A, this operation may require polishing (e.g., blunting) the ends of the cfDNA with a polymerase, A-tailing the fragments using, e.g., Taq polymerase, and ligating a T-tailed Y-adapters to the A-tailed fragments. This initial ligation operation may be performed on a limiting amount of cfDNA. For example, cfDNA to which the adapters are ligated may contain less than 200 ng of DNA, e.g., 10 pg to 200 ng, 100 pg to 200 ng, 1 ng to 200 ng, 5 ng to 50 ng, or less than 10,000 ng (e.g., less than 5,000, less than 1,000, less than 500, less than 100, or less than 10) haploid genome equivalents, depending on the genome. In some embodiments, the method is performed using less than 50 ng of cfDNA (which roughly corresponds to approximately 5 mL of plasma) or less than 10 ng of cfDNA, which roughly corresponds to approximately 1 mL of plasma. For example, Newman et al. (“An ultrasensitive method for quantitating circulating tumor DNA with broad patient coverage”, Nat Med. 2014 20: 548-54, which is incorporated by reference herein) describes libraries from 7-32 ng cfDNA isolated from 1-5 mL plasma. This is equivalent to 2,121-9,697 haploid genomes (assuming 3.3 pg per haploid genome). The adapters ligated onto the cfDNA may contain a molecular barcode to facilitate multiplexing and quantitative analysis of the sequenced molecules. Specifically, the adapters may be “indexed” in that the adapters contain a molecular barcode that identifies the sample to which the sample was ligated, which allows samples to be pooled before sequencing. Alternatively, or additionally, the adapters may contain a random barcode or the like. Such an adapters can be ligated to the fragments and substantially every fragment corresponding to a particular region are tagged with a different sequence. This allows for identification of PCR duplicates and allows molecules to be counted.

In the next operation of this implementation of the method, the hydroxymethylated DNA molecules in the cfDNA are labeled with a with the chemoselective group, e.g., a group that can participate in a click reaction. This operation may be done by incubating the adapter-ligated cfDNA with DNA β-glucosyltransferase (e.g., T4 DNA β-glucosyltransferase (which is commercially available from a number of vendors), although other DNA β-glucosyltransferases exist) and, e.g., UDP-6-N3-GIU (e.g., UDP glucose containing an azide). This operation may be done using a protocol adapted from U.S. Patent Publication No. US20110301045, which is incorporated by reference herein, or Song et al., (“Selective chemical labeling reveals the genome-wide distribution of 5-hydroxymethylcytosine”, Nat. Biotechnol. 2011 29: 68-72, which is incorporated by reference herein), for example.

The next operation of this implementation of the method involves adding a biotin moiety to the chemoselectively modified DNA via a cycloaddition (click) reaction. This operation may be done by directly adding a biotinylated reactant, e.g., a dibenzocyclooctyne-modified biotin to the glucosyltransferase reaction after that reaction has been completed, e.g., after an appropriate amount of time (e.g., after 30 minutes or more). In some embodiments, the biotinylated reactant may be of the general formula B-L-X, where B is a biotin moiety, L is a linker and X is a group that reacts with the chemoselective group added to the cfDNA via a cycloaddition reaction. In certain cases, the linker may make the compound more soluble in an aqueous environment and, as such, may contain a polyethyleneglycol (PEG) linker or an equivalent thereof. In some embodiments, the added compound may be dibenzocyclooctyne-PEGn-biotin, where Nis 2-10, e.g., 4. Dibenzocyclooctyne-PEG4-biotin is relatively hydrophilic and is soluble in aqueous buffer up to a concentration of 0.35 mM. The compound added in this operation does not need to contain a cleavable linkage, e.g., does not contain a disulfide linkage or the like. In this operation, the cycloaddition reaction may be between an azido group added to the hydroxymethylated cfDNA and an alkynyl group (e.g., dibenzocyclooctyne group) that is linked to the biotin moiety. Again, this operation may be done using a protocol adapted from U.S. Patent Publication No. US20110301045 or Song et al., (“Selective chemical labeling reveals the genome-wide distribution of 5-hydroxymethylcytosine”, Nat. Biotechnol. 2011 29: 68-72, which is incorporated by reference herein), for example.

The enrichment operation of the method may be done using magnetic streptavidin beads, although other supports may be used. As noted above, the enriched cfDNA molecules (which correspond to the hydroxymethylated cfDNA molecules) are amplified by PCR and then sequenced.

In these embodiments, the enriched DNA sample may be amplified using one or more primers that hybridize to the added adapters (or their complements). In embodiments in which Y-adapters are added, the adapters-ligated nucleic acids may be amplified by PCR using two primers: a first primer that hybridizes to the single-stranded region of the top strand of the adapters, and a second primer that hybridizes to the complement of the single-stranded region of the bottom strand of the Y-adapters (or hairpin adapters, after cleavage of the loop). For example, in some embodiments the Y-adapters used may have P5 and P7 arms (e.g., with sequences that are compatible with Illumina sequencing platforms) and the amplification products may have the P5 sequence at one and the P7 sequence at the other. These amplification products can be hybridized to an Illumina sequencing substrate and sequenced. In some embodiments, the pair of primers used for amplification may have 3′ ends that hybridize to the Y-adapters and 5′ tails that either have the P5 sequence or the P7 sequence. In these embodiment, the amplification products may also have the P5 sequence at one and the P7 sequence at the other. These amplification products can be hybridized to an Illumina sequencing substrate and sequenced. This amplification operation may be performed by limited cycle PCR (e.g., 5-20 cycles).

The sequencing operation may be done using any convenient next generation sequencing method and may result in at least 10,000, at least 50,000, at least 100,000, at least 500,000, at least 1 million, at least 10 million, at least 100 million, or at least 1 billion sequence reads. In some cases, the reads are paired-end reads. The primers may be used for amplification and may be compatible with use in any next generation sequencing platform in which primer extension is used, e.g., Illumina's reversible terminator method, Roche's pyrosequencing method (454), Life Technologies' sequencing by ligation (the SOLID platform), Life Technologies' Ion Torrent platform or Pacific Biosciences' fluorescent base-cleavage method. Examples of such methods are described in the following references: Margulies et al. (“Genome sequencing in microfabricated high-density picolitre reactors”, Nature 2005; 437:376-380); Ronaghi et al. (“Real-time DNA sequencing using detection of pyrophosphate release”, Anal Biochem. 1996; 242:84-89); Shendure et al. (“Accurate Multiplex Polony Sequencing of an Evolved Bacterial Genome”, Science 2005; 309:1728-1732); Imelfort et al. (“De novo sequencing of plant genomes using second-generation technologies”, Brief Bioinform. 2009; 10:609-618); Fox et al. (“Applications of ultra-high-throughput sequencing”, Methods Mol Biol. 2009; 553:79-108); Appleby et al. (“New technologies for ultra-high throughput genotyping in plants”, Methods Mol Biol. 2009; 513:19-39); English et al. (“Mind the Gap: Upgrading Genomes with Pacific Biosciences RS Long-Read Sequencing Technology”, PLoS ONE. 2012; 7:e47768); and Morozova et al. (“Applications of next-generation sequencing technologies in functional genomics”, Genomics. 2008; 92:255-264), each of which is incorporated by reference herein, and may be used for the general descriptions of the methods and the particular operations of the methods, including all starting products, reagents, and final products for each of the operations.

In some embodiments, the sample sequenced may comprise a pool of DNA molecules from a plurality of samples in which the nucleic acids in the sample contain a molecular barcode to indicate their source. In some embodiments, the nucleic acids may be derived from a single source (e.g., a single organism, virus, tissue, cell, subject, etc.). In other embodiments, the nucleic acid sample may be a pool of nucleic acids extracted from a plurality of sources (e.g., a pool of nucleic acids from a plurality of organisms, tissues, cells, subjects, etc.), whereby “plurality” means two or more. As such, in some embodiments, a nucleic acid sample can contain nucleic acids from 2 or more sources, 3 or more sources, 5 or more sources, 10 or more sources, 50 or more sources, 100 or more sources, 500 or more sources, 1000 or more sources, 5000 or more sources, up to and including about 10,000 or more sources. Molecular barcodes may allow the sequences from different sources to be distinguished after they are analyzed.

The sequence reads may be analyzed by a computer and, as such, instructions for performing the operations set forth below may be set forth as programing that may be recorded in a suitable physical computer readable storage medium.

### II. Computer Systems and Machine Learning Methods

### A. Sample Features

As used herein, relating to machine learning and pattern recognition, the term “feature” may refer to an individual measurable property or characteristic of a phenomenon being observed. Features may be numeric, but structural features, such as strings and graphs, may be used in syntactic pattern recognition. The concept of “feature” may be related to that of explanatory variable used in statistical techniques such as linear regression.

In some embodiments, the hydroxymethylation state data are featurized and processed using a trained machine learning model that is trained to classify the sample into groups according to predesignated or preselected biological properties.

In some embodiments, a set of features is identified from the nucleic acid sequences to be processed using a machine learning model. The set of features can correspond to properties of the nucleic acid sequences in the biological sample.

In some embodiments, the properties of the nucleic acid sequences are selected from the presence or absence of cancer or a stage of cancer, or a prognosis of cancer in an individual from whom the sample was obtained.

The training samples can be selected based on the desired classification, e.g., as indicated by a clinical question. Different subsets can have different properties, e.g., as determined by labels assigned to the subsets. A first subset of the training biological samples can be identified as having a specified property and a second subset of the training biological samples can be identified as not having the specified property. Examples of properties may be various diseases or disorders but may be intermediate classifications or measurements as well. Examples of such properties include, but are limited to, the existence of cancer or a stage of cancer, or a prognosis of cancer, e.g., if untreated or in response to a treatment of the cancer. As examples, the cancer can be colorectal cancer, liver cancer, lung cancer, pancreatic cancer, or breast cancer.

In some embodiments, the features are processed using a feature matrix for machine learning analysis.

For a plurality of assays, the system may identify feature sets to be processed using a machine learning model. The system may perform an assay on each molecule class and form a feature vector from the measured values. The system may process the feature vector using the machine learning model and obtain an output classification of whether the biological sample has a specified property.

In some embodiments, the machine learning model outputs a classifier that distinguishes between two groups or classes of individuals or features in a population of individuals or features of the population. In some embodiments, the classifier is a trained machine learning classifier.

In some embodiments, the informative loci or features of biomarkers in a cancer tissue are assayed to form a profile. Receiver Operating Characteristic (ROC) curves may be useful for plotting the performance of a particular feature (e.g., any of the biomarkers described herein and/or any item of additional biomedical information) in distinguishing between two populations (e.g., individuals responding and not responding to a therapeutic agent). The feature data across the entire population (e.g., the cases and controls) may be sorted in ascending order based on the value of a single feature.

In some embodiments, the condition is advanced adenoma (AA), colorectal cancer (CRC), colorectal carcinoma, or inflammatory bowel disease.

The term “input features” or “features” may refer to variables that are used by the model to predict an output classification (label) of a sample, e.g., a condition, sequence content (e.g., mutations), suggested data collection operations, or suggested treatments. Values of the variables can be determined for a sample and used to determine a classification. Example of input features of genetic data include: aligned variables that relate to alignment of sequence data (e.g., sequence reads) to a genome and non-aligned variables, e.g., that relate to the sequence content of a sequence read, a measurement of protein or autoantibody, or the mean methylation level at a genomic region.

In various embodiments, hydroxymethylation status in a nucleic acid sequence may be featurized to include: 1) single CpG site features (e.g., ratio of 5hmC to C or % hydroxymethylation), ratio of 5hmC to 5mC, ratio of 5hmC to total methylation (5mC+5hmC) for CpG sites; 2) single CH site (e.g., ratio of 5hmC to C or % hydroxymethylation), ratio of 5hmC to 5mC, ratio of 5hmC to total methylation (5mC+5hmC) for CH sites); 3) fragment-level 5hmC features (e.g., calling a cfDNA fragment as hydroxymethylated if the fragment has ≥X 5hmC CpG sites, calling a cfDNA fragment as hydroxymethylated if ≥X % of CpG sites are 5hmC, calling a cfDNA fragment as hydroxymethylated if the fragment has ≥X 5hmC sites (not just CpG), calling a cfDNA fragment as hydroxymethylated if ≥X % of C's (not just CpG sites) are 5hmC over each fragment); and 4) region-level 5hmC features (e.g., calling a cfDNA fragment as hydroxymethylated if the fragment has ≥X 5hmC CpG sites, calling a cfDNA fragment as hydroxymethylated if ≥X % of CpG sites are 5hmC, calling a cfDNA fragment as hydroxymethylated if the fragment has ≥X 5hmC sites (not just CpG), calling a cfDNA fragment as hydroxymethylated if ≥X % of C's (not just CpG sites) are 5hmC over each gene body and for each gene body, featurize to include ratio of 5hmC to C or % hydroxymethylation), ratio of 5hmC to 5mC, ratio of 5hmC to total methylation (5mC+5hmC), or a combination thereof, where X is any number.

In some embodiments, featurizing across a gene body sequence may include exons only (e.g., by aggregating together all exons for a given gene), transcription start site region (e.g., 1-kb region surrounding the TSS), enhancers, CpG shelves, CpG shores, or CpG islands.

Values of the variables can be determined for a sample and used to determine a classification. Example of input features of genetic data include: aligned variables that relate to alignment of sequence data (e.g., sequence reads) to a genome and non-aligned variables, e.g., that relate to the sequence content of a sequence read, a measurement of protein or autoantibody, or the mean methylation level at a genomic region. In various examples, genetic features such as, V-plot measures, transcription factor binding analysis, FREE-C deconvolution, the cfDNA measurement over a transcription start site and DNA hydroxymethylation levels over cfDNA fragments may be used as input features to be processed by machine learning methods and models.

In some examples, the sequencing information includes information regarding a plurality of genetic features such as, but not limited to, transcription start sites, transcription factor binding sites, chromatin open and closed states, nucleosomal positioning or occupancy, and the like.

### B. Data Analysis

In some embodiments, the present disclosure provides a system, method, or kit having data analysis realized in software applications, computing hardware, or both. In various embodiments, the analysis application or system includes at least a data receiving module, a data pre-processing module, a data analysis module (which can operate on one or more types of genomic data), a data interpretation module, or a data visualization module. In some embodiments, the data receiving module can comprise computer systems that connect laboratory hardware or instrumentation with computer systems that process laboratory data. In some embodiments, the data pre-processing module can comprise hardware systems or computer software that performs operations on the data in preparation for analysis. Examples of operations that can be applied to the data in the pre-processing module include affine transformations, denoising operations, data cleaning, reformatting, or subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

In various embodiments, machine learning methods are applied to distinguish samples in a population of samples. In some embodiments, machine learning methods are applied to distinguish samples between healthy and advanced adenoma samples.

In some embodiments, the one or more machine learning operations used to train the methylation-based prediction engine include one or more of: a generalized linear model, a generalized additive model, a non-parametric regression operation, a random forest classifier, a spatial regression operation, a Bayesian regression model, a time series analysis, a Bayesian network, a Gaussian network, a decision tree learning operation, an artificial neural network, a recurrent neural network, a reinforcement learning operation, linear/non-linear regression operations, a support vector machine, a clustering operation, and a genetic algorithm operation.

In various embodiments, computer processing methods are selected from logistic regression, multiple linear regression (MLR), dimension reduction, partial least squares (PLS) regression, principal component regression, autoencoders, variational autoencoders, singular value decomposition, Fourier bases, wavelets, discriminant analysis, support vector machine, decision tree, classification and regression trees (CART), tree-based methods, random forest, gradient boost tree, logistic regression, matrix factorization, multidimensional scaling (MDS), dimensionality reduction methods, t-distributed stochastic neighbor embedding (t-SNE), multilayer perceptron (MLP), network clustering, neuro-fuzzy, and artificial neural networks.

In some embodiments, the methods disclosed herein can include computational analysis on nucleic acid sequencing data of samples from an individual or from a plurality of individuals. An analysis can identify a variant inferred from sequence data to identify sequence variants based on probabilistic modeling, statistical modeling, mechanistic modeling, network modeling, or statistical inferences. Non-limiting examples of analysis methods include principal component analysis, autoencoders, singular value decomposition, Fourier bases, wavelets, discriminant analysis, regression, support vector machines, tree-based methods, networks, matrix factorization, and clustering. Non-limiting examples of variants include a germline variation or a somatic mutation. In some embodiments, a variant can refer to an observed variant. The observed variant can be scientifically confirmed or reported in literature. In some embodiments, a variant can refer to a putative variant associated with a biological change. A biological change can be observed or unobserved (e.g., known or unknown). In some embodiments, a putative variant can be reported in literature, but not yet biologically confirmed.

Alternatively, a putative variant may not be reported in literature, but can be inferred based on a computational analysis disclosed herein. In some embodiments, germline variants can refer to nucleic acids that induce natural or normal variations.

Natural or normal variations can include, for example, skin color, hair color, and normal weight. In some embodiments, somatic mutations can refer to nucleic acids that induce acquired or abnormal variations. Acquired or abnormal variations can include, for example, cancer, obesity, conditions, symptoms, diseases, and disorders. In some embodiments, the analysis can include distinguishing between germline variants. Germline variants can include, for example, private variants and somatic mutations. In some embodiments, the identified variants can be used by clinicians or other health professionals to improve health care methodologies, accuracy of diagnoses, and cost reduction.

Also provided herein are improved methods and computing systems or software media that can distinguish among sequence errors in nucleic acid introduced through amplification and/or sequencing techniques, somatic mutations, and germline variants. Methods provided can include simultaneously calling and scoring variants from aligned sequencing data of all samples obtained from a patient.

Samples obtained from subjects other than the patient can also be used. Other samples can also be collected from subjects previously analyzed by a sequencing assay or a targeted sequencing assay (e.g., a targeted resequencing assay). Methods, computing systems, or software media disclosed herein can improve identification and accuracy of variations or mutations (e.g., germline or somatic, including copy number variations, single nucleotide variations, indels, a gene fusions), and lower limits of detection by reducing the number of false positive and false negative identifications.

### C. Classifier Generation

In some aspects, the present systems and methods provide a classifier generated based on feature information derived from methylation sequence analysis from biological samples of cfDNA. The classifier may form part of a predictive engine for distinguishing groups in a population based on methylation sequence features identified in biological samples such as cfDNA.

In some embodiments, a classifier is created by normalizing the methylation information by formatting similar portions of the methylation information into a unified format and a unified scale; storing the normalized methylation information in a columnar database; training a methylation prediction engine by applying one or more one machine learning operations to the stored normalized methylation information, the methylation prediction engine mapping, for a particular population, a combination of one or more features; applying the methylation prediction engine to the accessed field information to identify a methylation associated with a group; and classifying the individual into a group.

Specificity may be defined as the probability of a negative test among those who are free from the disease. Specificity is equal to the number of disease-free persons who tested negative divided by the total number of disease-free individuals.

In various embodiments, the model, classifier, or predictive test has a specificity of at least 40%, at least 45%, at least 50%, at least 55%, at least 60%, at least 65%, at least 70%, at least 75%, at least 80%, at least 85%, at least 90%, at least 95%, or at least 99%.

Sensitivity may be defined as the probability of a positive test among those who have the disease. Sensitivity is equal to the number of diseased individuals who tested positive divided by the total number of diseased individuals.

In various embodiments, the model, classifier, or predictive test has a sensitivity of at least 40%, at least 45%, at least 50%, at least 55%, at least 60%, at least 65%, at least 70%, at least 75%, at least 80%, at least 85%, at least 90%, at least 95%, or at least 99%.

In some embodiments, the group is healthy (asymptomatic), inflammatory bowel disease, AA, or CRC.

### D. Digital Processing Device

In some embodiments, described herein is a digital processing device or use of the same. In some embodiments, the digital processing device can include one or more hardware central processing units (CPU), graphics processing units (GPU), or tensor processing units (TPU) that carry out the device's functions. In some embodiments, the digital processing device can include an operating system configured to perform executable instructions. In some embodiments, the digital processing device can optionally be connected a computer network. In some embodiments, the digital processing device can be optionally connected to the Internet such that the device accesses the World Wide Web. In some embodiments, the digital processing device can be optionally connected to a cloud computing infrastructure. In some embodiments, the digital processing device can be optionally connected to an intranet. In some embodiments, the digital processing device can be optionally connected to a data storage device.

Non-limiting examples of suitable digital processing devices include server computers, desktop computers, laptop computers, notebook computers, sub-notebook computers, netbook computers, netpad computers, set-top computers, handheld computers, Internet appliances, mobile smartphones, and tablet computers. Suitable tablet computers can include, for example, those with booklet, slate, and convertible configurations.

In some embodiments, the digital processing device can include an operating system configured to perform executable instructions. For example, the operating system can include software, including programs and data, which manages the device's hardware and provides services for execution of applications. Non-limiting examples of operating systems include Ubuntu, FreeBSD, OpenBSD, NetBSD®, Linux, Apple® Mac OS X Server®, Oracle® Solaris®, Windows Server®, and Novell® NetWare®. Non-limiting examples of suitable personal computer operating systems include Microsoft® Windows®, Apple® Mac OS X®, UNIX®, and UNIX-like operating systems such as GNU/Linux®. In some embodiments, the operating system can be provided by cloud computing, and cloud computing resources can be provided by one or more service providers.

In some embodiments, the device can include a storage and/or memory device. The storage and/or memory device can be one or more physical apparatuses used to store data or programs on a temporary or permanent basis. In some embodiments, the device can be volatile memory and require power to maintain stored information. In some embodiments, the device can be non-volatile memory and retain stored information when the digital processing device is not powered. In some embodiments, the non-volatile memory can include flash memory. In some embodiments, the non-volatile memory can include dynamic random-access memory (DRAM). In some embodiments, the non-volatile memory can include ferroelectric random access memory (FRAM). In some embodiments, the non-volatile memory can include phase-change random access memory (PRAM). In some embodiments, the device can be a storage device including, for example, CD-ROMs, DVDs, flash memory devices, magnetic disk drives, magnetic tapes drives, optical disk drives, and cloud computing-based storage. In some embodiments, the storage and/or memory device can be a combination of devices such as those disclosed herein.

In some embodiments, the digital processing device can include a display to send visual information to a user. In some embodiments, the display can be a cathode ray tube (CRT). In some embodiments, the display can be a liquid crystal display (LCD). In some embodiments, the display can be a thin film transistor liquid crystal display (TFT-LCD). In some embodiments, the display can be an organic light emitting diode (OLED) display. In some embodiments, on OLED display can be a passive-matrix OLED (PMOLED) or active-matrix OLED (AMOLED) display. In some embodiments, the display can be a plasma display. In some embodiments, the display can be a video projector. In some embodiments, the display can be a combination of devices such as those disclosed herein.

In some embodiments, the digital processing device can include an input device to receive and process information from a user. In some embodiments, the input device can be a keyboard. In some embodiments, the input device can be a pointing device including, for example, a mouse, trackball, track pad, joystick, game controller, or stylus. In some embodiments, the input device can be a touch screen or a multi-touch screen. In some embodiments, the input device can be a microphone to capture voice or other sound input. In some embodiments, the input device can be a video camera to capture motion or visual input. In some embodiments, the input device can be a combination of devices such as those disclosed herein.

### E. Non-Transitory Computer-Readable Storage Medium

In some embodiments, the subject matter disclosed herein can include one or more non-transitory computer-readable storage media encoded with a program including instructions executable by the operating system of an optionally networked digital processing device. In some embodiments, a computer-readable storage medium can be a tangible component of a digital processing device. In some embodiments, a computer-readable storage medium can be optionally removable from a digital processing device. In some embodiments, a computer-readable storage medium can include, for example, CD-ROMs, DVDs, flash memory devices, solid state memory, magnetic disk drives, magnetic tape drives, optical disk drives, cloud computing systems and services, and the like. In some embodiments, the program and instructions can be permanently, substantially permanently, semi-permanently, or non-transitorily encoded on the media.

### F. Computer Systems

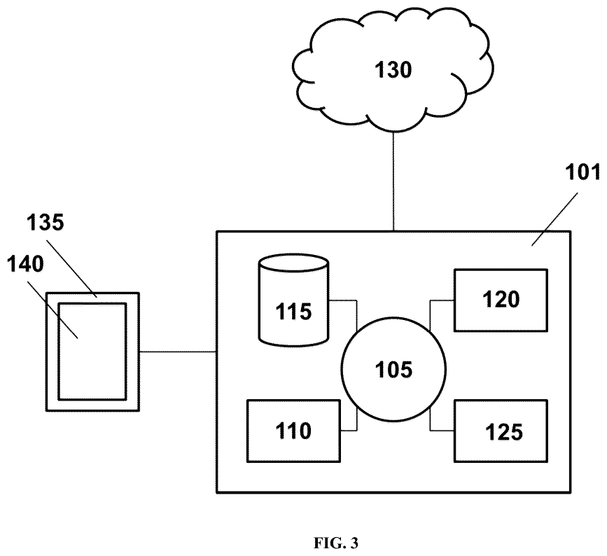

The present disclosure provides computer systems that are programmed to implement methods of the disclosure. FIG. 3 shows a computer system 101 that is programmed or otherwise configured to store, process, identify, or interpret patient data, biological data, biological sequences, or reference sequences. The computer system 101 can process various aspects of patient data, biological data, biological sequences, or reference sequences of the present disclosure. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single-core or multi-core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage, and/or electronic display adapters. The memory 110, storage unit 115, interface 120, and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some embodiments is a telecommunication and/or data network. The network 130 can include one or more computer servers, which can enable distributed computing, such as cloud computing. The network 130, in some embodiments with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some embodiments, the circuit is an application specific integrated circuit (ASIC).

The storage unit 115 can store files, such as drivers, libraries, and saved programs. The storage unit 115 can store user data, e.g., user preferences and user programs. The computer system 101, in some embodiments, can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PCs (e.g., Apple® iPad, Samsung® Galaxy Tab), telephones, smart phones (e.g., Apple® iphone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some embodiments, the code can be retrieved from the storage unit 115 and stored on the memory 110 for ready access by the processor 105. In some embodiments, the electronic storage unit 115 can be precluded, and machine-executable instructions are stored on memory 110.

The code can be pre-compiled and configured for use with a machine having a processer adapted to execute the code or can be interpreted or compiled during runtime. The code can be supplied in a programming language that can be selected to enable the code to execute in a pre-compiled, interpreted, or as-compiled fashion.

Aspects of the systems and methods provided herein, such as the computer system 101, can be embodied in programming. Various aspects of the technology may be considered “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

### III. Methods of Use

### A. Disease Detection and Diagnosis

Methods and systems provided herein may perform predictive analytics using artificial intelligence-based approaches to analyze acquired data from a subject (patient) to generate an output of diagnosis of the subject having a cancer (e.g., CRC). For example, the application may apply a prediction algorithm to the acquired data to generate the diagnosis of the subject having the cancer. The prediction algorithm may comprise an artificial intelligence-based predictor, such as a machine learning-based predictor, configured to process the acquired data to generate the diagnosis of the subject having the cancer.

In some embodiments, the cancer detected or assessed using products or processes described herein includes, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, and Burkitt's lymphoma.

In some embodiments, the products or processes described herein may be used to detect or assess a premalignant condition, such as actinic keratosis, atrophic gastritis, leukoplakia, erythroplasia, lymphomatoid granulomatosis, preleukemia, fibrosis, cervical dysplasia, uterine cervical dysplasia, xeroderma pigmentosum, Barrett's esophagus, colorectal polyp, or other abnormal tissue growth or lesion that is likely to develop into a malignant tumor. Transformative viral infections, such as HIV and HPV, also present phenotypes that may be assessed according to the method.

The cancer characterized by the present method may be, without limitation, a carcinoma, a sarcoma, a lymphoma or leukemia, a germ cell tumor, a blastoma, or other cancers. Carcinomas include, without limitation, epithelial neoplasms, squamous cell neoplasms, squamous cell carcinoma, basal cell neoplasms, basal cell carcinoma, transitional cell papillomas and carcinomas, adenomas and adenocarcinomas (glands), adenoma, adenocarcinoma, linitis plastica, insulinoma, glucagonoma, gastrinoma, vipoma, cholangiocarcinoma, hepatocellular carcinoma, adenoid cystic carcinoma, carcinoid tumor of appendix, prolactinoma, oncocytoma, Hurthle cell adenoma, renal cell carcinoma, Grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, Warthin tumor, thymoma, specialized gonadal neoplasms, sex cord stromal tumor, thecoma, granulosa cell tumor, arrhenoblastoma, Sertoli-leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes, without limitation, Askin's tumor, botryoides, chondrosarcoma, Ewing's sarcoma, malignant hemangioendothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma. Lymphoma and leukemia include, without limitation, chronic lymphocytic leukemia/small lymphocytic lymphoma, B-cell prolymphocytic leukemia, lymphoplasmacytic lymphoma (such as Waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, Mycosis fungoides/Sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma. Germ cell tumors include, without limitation, germinoma, dysgerminoma, seminoma, nongerminomatous germ cell tumor, embryonal carcinoma, endodermal sinus tumor, choriocarcinoma, teratoma, polyembryoma, and gonadoblastoma. Blastoma includes, without limitation, nephroblastoma, medulloblastoma, and retinoblastoma. Other cancers include, without limitation, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidla melanoma, seminoma, rhabdomyosarcoma, craniopharyngioma, osteosarcoma, chondrosarcoma, myosarcoma, liposarcoma, fibrosarcoma, Ewing sarcoma, and plasmacytoma.

In a further embodiment, the cancer under analysis may be a lung cancer, including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or a solid tumor.

In further embodiments, the cancer may be an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer, renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilm's tumor. The methods of the present disclosure can be used to characterize these and other cancers. Thus, characterizing a phenotype can be providing a diagnosis, prognosis, or theranosis of one of the cancers disclosed herein.

The machine learning predictor may be trained using datasets, e.g., datasets generated by performing multi-analyte assays of biological samples of individuals, from one or more sets of cohorts of patients having cancer as inputs and a clinical diagnosis (e.g., staging and/or tumor fraction) outcomes of the subjects as outputs to the machine learning predictor.

Training datasets (e.g., datasets generated by performing multi-analyte assays of biological samples of individuals) may be generated from, for example, one or more sets of subjects having common characteristics (features) and outcomes (labels). Training datasets may comprise a set of features and labels corresponding to the features relating to diagnosis. Features may comprise characteristics such as, for example, certain ranges or categories of cfDNA assay measurements, such as counts of cfDNA fragments in a biological sample obtained from a healthy and disease samples that overlap or fall within each of a set of bins (genomic windows) of a reference genome. For example, a set of features collected from a given subject at a given time point may collectively serve as a diagnostic signature, which may be indicative of an identified cancer of the subject at the given time point. Characteristics may also include labels indicating the subject's diagnostic outcome, such as for one or more cancers.

Labels may comprise outcomes such as, for example, a clinical diagnosis (e.g., staging and/or tumor fraction) outcomes of the subject. Outcomes may include a characteristic associated with the cancers in the subject. For example, characteristics may be indicative of the subject having one or more cancers.

Training sets (e.g., training datasets) may be selected by random sampling of a set of data corresponding to one or more sets of subjects (e.g., retrospective and/or prospective cohorts of patients having or not having one or more cancers). Alternatively, training sets (e.g., training datasets) may be selected by proportionate sampling of a set of data corresponding to one or more sets of subjects (e.g., retrospective and/or prospective cohorts of patients having or not having one or more cancers). Training sets may be balanced across sets of data corresponding to one or more sets of subjects (e.g., patients from different clinical sites or trials). The machine learning predictor may be trained until certain pre-determined conditions for accuracy or performance are satisfied, such as having minimum desired values corresponding to diagnostic accuracy measures. For example, the diagnostic accuracy measure may correspond to prediction of a diagnosis, staging, or tumor fraction of one or more cancers in the subject.

Examples of diagnostic accuracy measures may include sensitivity, specificity, PPV, NPV, accuracy, and AUC of a ROC curve corresponding to the diagnostic accuracy of detecting or predicting the cancer (e.g., colorectal cancer).

In another aspect, the present disclosure provides a method for identifying a cancer in a subject, the method comprising: (a) providing a biological sample comprising cell-free nucleic acid (cfNA) molecules from said subject; (b) methylation sequencing said cfNA molecules from said subject to generate a plurality of cfNA sequencing reads; (c) aligning said plurality of cfNA sequencing reads to a reference genome; (d) generating a quantitative measure of said plurality of cfNA sequencing reads at each of a first plurality of genomic regions of said reference genome to generate a first cfNA feature set, wherein said first plurality of genomic regions of said reference genome comprises at least about 10 distinct regions, each of said at least about 10 distinct regions; and (e) applying a trained algorithm to said first cfNA feature set to generate a likelihood of said subject having said cancer.

In some embodiments, the method may include comparing measured hydroxymethylation levels in predetermined regions of interest (ROIs) from the subject at risk of having a disease or cell proliferation disorder against a database of measured hydroxymethylation levels in normal or healthy subjects for analogous predetermined ROIs; and determining that the subject has an increased risk of having a cellular proliferation disorder by quantifying differentially hydroxymethylated nucleic acid fragments in predetermined ROIs of the subject compared to predetermined ROIs of normal or healthy subjects in the database of measured hydroxymethylation levels in normal or healthy subjects for analogous predetermined ROIs.

For example, such a pre-determined condition may be that the sensitivity of predicting the cancer (e.g., colorectal cancer, breast cancer, pancreatic cancer, or liver cancer) comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the cancer (e.g., colorectal cancer, breast cancer, pancreatic cancer, or liver cancer) comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the PPV of predicting the cancer (e.g., colorectal cancer, breast cancer, pancreatic cancer, or liver cancer) comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the NPV of predicting the cancer (e.g., colorectal cancer, breast cancer, pancreatic cancer, or liver cancer) comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the AUC of a ROC curve of predicting the cancer (e.g., colorectal cancer, breast cancer, pancreatic cancer, or liver cancer) comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some examples of any of the foregoing aspects, a method further comprises monitoring a progression of a disease in the subject, wherein the monitoring is based at least in part on the genetic sequence feature. In some examples, the disease is a cancer.

In some embodiments, methods described here are useful to determine the contribution of 5-hydroxymethylation signal to total methylation signal in patient samples. Total methylation signal may be derived from various sequencing methods including bisulfite or enzymatic based library preparation for methylation detection. Contributions of 5hmC to noise that negatively impacts the sensitivity or specificity of diagnosis may be removed from the total methylation signal to improve test performance.

In some embodiments, methods described here are useful for 5hmC detection can be used in a similar manner to oxidative bisulfite sequencing (oxBS-seq). Conversion of C, 5hmC, 5fC, and 5caC bases to uracil without conversion of 5mC may allow detection of only 5mC. 5hmC signal can be subtracted from total methylation signal to achieve a “true methyl” signal at base resolution, but using lower DNA inputs. Subtraction of 5hmC from total methylation signal provides a readout of a “true methyl” or 5mC signal in DNA. oxBS-seq may entail chemical oxidation of 5hmC to 5fC followed by bisulfite conversion requiring high DNA inputs.

In some embodiments, methods described here are useful for analysis of nucleotide resolution 5hmC alone or in combination with total methylation signal to improve prediction of gene expression. Features for prediction may include per CpG or fragment level 5hmC levels and 5hmC/5mC ratios at relevant genome features such as promoters, enhancers, UTRs, and gene bodies.

In some embodiments, methods described here are useful to collect nucleotide-level 5hmC signatures in various tissues, cell types, and cancer types, thereby increasing the resolution of past 5hmC tissue maps. Analysis of these data may be used for more sensitive and specific determination of tissue of origin for cancer diagnosis and prognosis.

In some embodiments, methods described here are useful for biomarker discovery for patient response to cancer treatment. Abundance of 5hmC signal in cfDNA or the presence of tissue-specific 5hmC signal can be used to track residual disease after treatment for one or more cancer types.

In some embodiments, methods described here may use cfDNA-derived 5hmC sequence data information at drug target genes for companion diagnostic methods to identify patients likely to respond or actively responding to drug treatment, effectiveness of patient response to a drug, or patients at risk of side-effects due to treatment.

### EXAMPLES

### Example 1: Use of Modified Oligonucleotide Adapters for Improved Resolution of 5hmC-Containing Nucleic Acids

The methods described herein can be used for generation of nucleotide-resolution 5hmC sequencing libraries from cell-free or genomic DNA molecules in patient samples. Libraries can be generated genome-wide or for targeted regions. Analysis of 5hmC DNA modifications may have many applications including biomarker discovery for cancer detection, tissue of origin determination, cancer prognosis, and companion diagnostic development. Featurized hydroxymethylation state data may be used as input for applications including hydroxymethylation profiling to identify biomarkers characteristic of disease (including subtype stratification) or to train a machine learning model useful to classify individual samples for disease detection.

### Methods

The enzymatic hydroxymethylation sequencing (EHM-seq) method for 5hmC detection may include the following operations:
a. Enzymatic oxidation and optionally glucosylation of 5mC adapters;
b End Preparation of input DNA;
c. Adapter ligation to input DNA using enzymatically oxidized adapters;
d. Protection of 5hmC by β-glucosylation and enzymatic deamination of C and 5mC to U in DNA molecules; and
e. Sequencing of converted input ligated DNA.

### A) The Enzymatic Oxidation of 5mC Adapters

Enzymatic oxidation of 5mC in adapters can include first enzymatically oxidizing to 5hmC, then to 5fC, and ultimately to 5caC, while in the same reaction glucosylating 5hmC to 5gmC. In this way, 5caC and 5gmC may be protected from downstream conversion to U.

The 5mC oxidation and glucosylation to 5caC and/or 5gmC protects adapters from downstream enzymatic conversion to U, which the ligated DNA molecule may be subjected to for 5hmC detection.

An alternative to enzymatically oxidizing 5mC adapters may be to synthesize 5hmC-containing adapters for use in a subsequent adapter ligation reaction.

### B) End Preparation Plus A-Tailing of Input DNA

End repair uses a DNA polymerase with 3′-5′ exonuclease activity to fill in 5′ overhangs and remove 3′ overhangs, thereby producing blunt ended DNA. A-tailing then attaches a single A nucleotide to the 3′ ends to allow for a subsequent high efficiency T/A-ligation operation. Alternatively, the A-tailing operation can be omitted if blunt-end ligation is used to attach adapters to DNA molecules.

### C) Adapter Ligation and Library Preparation

Enzymatically oxidized adapters are added to the adapter ligation reaction with sample DNA molecules at a final concentration of 1 μM. After adapter ligation, a clean-up is performed, and adapter-ligated DNA molecules are eluted in a final volume.

### D) Protection by Glucosylation of 5hmC to 5gmC

Ligated DNA is glucosylated. After glucosylation, a clean-up is performed and glucosylated adapter-ligated DNA molecules are eluted in a final volume.

The cleaned-up β-GT protected DNA is denatured followed by immediate incubation on ice. The denatured DNA is subjected to APOBEC reaction conditions to complete enzymatic conversion.

Converted DNA can then be PCR amplified and taken through target-enrichment and/or sequenced.

### Hydroxymethylation Analysis/Featurization

5hmC is preferentially represented at genic regions of the genome, including enhancers, promoters, and gene bodies. A useful featurization of data generated by the method described herein is used to calculate an aggregate 5hmC metric over gene bodies, such as the mean hydroxymethylation level (the number of hydroxymethylated CpGs detected overlapping a gene body divided by the total number of CpGs overlapping the gene body). One possible application of this metric is in classifying the disease state of a sample.

Analysis of cytosine methylation and hydroxymethylation in mammalian genomes has traditionally focused on methylation of cytosines in the CpG context, as CpG methylation constitutes the large majority of cytosine methylation in mammals. However, non-CpG methylation, namely CH methylation, may be biologically functional. Hydroxymethyl status in a nucleic acid sequence may be featurized to include the mean CH hydroxymethylation level over gene bodies. Once featurized, hydroxymethylation state data may be processed for applications including hydroxymethylation profiling to identify biomarkers characteristic of disease (including subtype stratification) or to train a machine learning model useful to classify individual samples for disease detection.

1.-72. (canceled)

73. A method for providing hydroxymethylation state data of nucleic acids in a biological sample, the method comprising:
a) obtaining the biological sample containing the nucleic acids;
b) ligating oligonucleotide adapters to at least a portion of the nucleic acids in the biological sample, wherein the oligonucleotide adapters comprise 5-hydroxymethylcytosine (5hmC) nucleotides, 5-(β-glucosyloxymethyl)cytosine (5gmC) nucleotides, 5-carboxycytosine (5caC) nucleotides, 5-carboxymethylcytosine (5cxmC) nucleotides, or a combination thereof, thereby generating ligated nucleic acids;
c) subjecting at least a portion of the ligated nucleic acids or a derivative thereof to a conversion condition that converts unmethylated and methylated cytosine nucleotides but not hydroxymethylated cytosine nucleotides of the ligated nucleic acids into uracil nucleotides, thereby generating converted nucleic acids; and
d) sequencing at least a portion of the converted nucleic acids to obtain a nucleic acid sequence of the converted nucleic acids, thereby providing the hydroxymethylation state data of the nucleic acids.

74. The method of claim 73, wherein the oligonucleotide adapters do not comprise cytosine nucleotides in flow cell binding regions or primer binding sites of the oligonucleotide adapters.

75. The method of claim 73, further comprising subjecting at least a portion of the ligated nucleic acids to glucosylation by β-glucosyltransferase (β-GT)/UDP-glucose to convert 5hmC nucleotides into 5gmC nucleotides after b) or prior to c).

76. The method of claim 73, wherein the conversion condition comprises bisulfite treatment, enzymatic treatment, or a combination thereof.

77. The method of claim 73, wherein the oligonucleotide adapters comprise 5hmC nucleotides.

78. The method of claim 73, wherein the oligonucleotide adapters comprise 5gmC nucleotides, 5caC nucleotides, 5cxmC nucleotides, or a combination thereof.

79. The method of claim 73, wherein the conversion condition comprises treatment with β-GT, a cytosine dioxygenase enzyme, carboxymethyltransferase, apolipoprotein B mRNA editing catalytic polypeptide-like protein (AID/APOBEC), or a combination thereof.

80. The method of claim 79, wherein the cytosine dioxygenase enzyme comprises ten eleven translocation protein 1 (TET1), ten eleven translocation protein 2 (TET2), ten eleven translocation protein 3 (TET3), or a functional variant thereof.

81. The method of claim 73, further comprising treating the oligonucleotide adapters with a TET enzyme after a) or prior to b).

82. The method of claim 73, further comprising performing a sequence enrichment after b) or prior to c).

83. The method of claim 82, wherein the sequence enrichment comprises a target capture hybridization.

84. The method of claim 73, further comprising amplifying at least a portion of the ligated nucleic acids prior to the sequencing.

85. The method of claim 73, wherein the oligonucleotide adapters are chemically synthesized using 5hmC phosphoramidites.

86. The method of claim 73, wherein the oligonucleotide adapters comprise 5gmC and 5caC nucleotides, and wherein the oligonucleotide adapters are produced at least in part by synthesizing 5mC-containing oligonucleotides using phosphoramidite chemistry and enzymatically treating the 5mC-containing oligonucleotides with a TET enzyme and β-GT/UDP-glucose.

87. The method of claim 73, further comprising aligning the nucleic acid sequence to a reference genome.

88. The method of claim 73, further comprising using the hydroxymethylation state data of the nucleic acids to train a machine learning model configured to generate hydroxymethylation state data.

89. The method of claim 73, wherein the biological sample is obtained or derived from a subject, and wherein the method further comprises processing the hydroxymethylation state data using a trained machine learning model configured to distinguish between subjects with the cell proliferative disorder and subjects without the cell proliferative disorder, and detecting a cell proliferative disorder in the subject based at least in part on the processing.

90. The method of claim 89, wherein the cell proliferative disorder comprises colorectal cancer, breast cancer, ovarian cancer, prostate cancer, lung cancer, pancreatic cancer, uterine cancer, liver cancer, esophagus cancer, stomach cancer, thyroid cancer, or bladder cancer.

91. The method of claim 89, wherein the trained machine learning model is tailored to detect the cell proliferative disorder at a pre-selected sensitivity and specificity.

92. The method of claim 89, further comprising featurizing the hydroxymethylation state data and processing the featurized hydroxymethylation state data using the trained machine learning model to classify the biological sample according to a pre-determined biological property of the subject.

93. The method of claim 92, wherein the pre-determined biological property comprises a presence or absence of pre-cancer, a presence or absence of cancer, a stage of cancer, or a prognosis of cancer in the subject.

94. A method for generating oligonucleotide adapters, the method comprising:
synthesizing oligonucleotides containing 5gmC nucleotides, 5caC nucleotides, 5cxmC nucleotides, or a combination thereof, at least in part by phosphoramidite chemistry, thereby generating the oligonucleotide adapters.
